(19) United States
(12) Patent Application Publication
(10) Pub. No.: US 2026/0207953 A1
(43) Pub. Date: Jul. 23, 2026

(54) OPTIMIZING CHEST COMPRESSION POSITION

(71) Applicant: Stryker Corporation, Portage, MI (US)

(72) Inventors: Erik von Schenck, Redmond, WA (US); David J. Linville, Redmond, WA (US); Tyson G. Taylor, Bothell, WA (US); Robert G. Walker, Seattle, WA (US)

(73) Assignee: Stryker Corporation, Portage, MI (US)

(21) Appl. No.: 19/451,624

(22) Filed: Jan. 16, 2026

Related U.S. Application Data
(60) Provisional application No. 63/746,897, filed on Jan. 17, 2025.

(51) Int. Cl. A61N 1/39; A61B 5/00; A61B 5/361; A61N 1/04; A61B 5/021; A61B 5/026; A61B 5/08; A61B 5/1455

(52) U.S. Cl. CPC A61N 1/39044; A61B 5/361; A61B 5/4836; A61N 1/046; A61N 1/3925; A61N 1/3987; A61N 1/3993; A61B 5/021; A61B 5/026; A61B 5/08; A61B 5/1455

(57) ABSTRACT

An example method includes detecting, by a sensor among an array of sensors in a chest-mounted apparatus, a compression applied to a chest of a subject by a compressor. By analyzing a position of the sensor among the array of sensors, a position of the compression is determined along a plane that is normal to an anterior-posterior direction. The example method further includes determining that the position of the compression is outside of a predetermined target range; and in response to determining that the position of the compression is outside of the predetermined target range, outputting an instruction to reposition the compressor.

### CROSS-REFERENCE TO RELATED APPLICATION

This application claims priority to and benefit of U.S. Provisional Patent Application No. 63/746,897 filed January 17, 2025, the entire contents of which are incorporated herein in their entirety.

### BACKGROUND

Cardiopulmonary resuscitation (CPR) is performed on subjects in respiratory and/or cardiac arrest. In various cases, administering CPR includes performing chest compressions. Chest compressions can induce blood flow in a subject whose heart is unable to spontaneously circulate blood. According to some examples, chest compressions can reduce the risk of the subject experiencing a serious and irreversible hypoxic injury until spontaneous circulation is restored.

In some cases, chest compressions are performed manually when a rescuer periodically pushes their hands onto the chest of the subject. In some examples, chest compressions are administered by a chest compression device. For instance, the chest compression device includes a compressor that periodically and precisely pushes the chest of the subject in order to administer chest compressions.

### BRIEF DESCRIPTION OF THE DRAWINGS

FIG. 1 illustrates an example environment for tracking chest compression position.

FIG. 2 illustrates an example of a chest-mounted apparatus disposed on the chest of a subject.

FIG. 3 illustrates an example circuit for detecting a position of a compression applied to an apparatus.

FIG. 4 illustrates an example cross-section of a portion of a chest-mounted apparatus.

FIG. 5 illustrates an example chest-mounted apparatus with selectable electrodes based on subject size.

FIG. 6 illustrates an example process for analyzing the position of chest compressions applied to a subject.

FIG. 7 illustrates an example process for adjusting the position of chest compressions applied to a subject.

FIG. 8 illustrates an example of an external defibrillator configured to perform various functions described herein.

FIG. 9 illustrates a chest compression device configured to perform various functions described herein.

FIG. 10 illustrates an example chest-mounted apparatus with for optimizing treatment based on vector selection and vector change.

### DETAILED DESCRIPTION

Chest compression efficacy depends significantly on the position of the compressing force. For example, the amount of blood flow induced by manual chest compressions depends on the placement of the rescuer’s hands on the subject’s chest. Moreover, the amount of blood flow induced by mechanical chest compressions depends on the placement of the compressor on the subject’s chest. Generally, blood flow can be maximized by applying chest compressions to a location at the center of the chest, such as at a location at the midline and between the nipples. However, for some subjects, blood flow is maximized when chest compressions are applied at slightly different locations, due to physiological variations within the population.

Improper chest compression placement can result in harm to the subject. For example, chest compressions applied to the subject at an incorrect position may result in inadequate blood perfusion, which can cause the subject to develop a hypoxic injury. In some cases, improperly placed chest compressions can physically harm the body of the subject. The risk of improperly placed chest compressions is particularly acute when chest compressions are administered or otherwise facilitated by untrained, or minimally trained, rescuers. However, even highly trained rescuers may apply chest compressions to an improper location on the subject’s chest, particularly at the scene of high-stress medical emergencies. Moreover, it may be difficult for any rescuer to predict an ideal chest compression placement on a subject that has unique physiological characteristics.

Various implementations described herein relate to techniques for optimizing a position at which chest compressions are applied to a subject. In various cases, a chest-mounted apparatus is configured to detect a position of a compression applied to the chest of the subject. The position, for instance, is with respect to the surface of the subject’s chest, with respect to a plane defined by superior-inferior direction and a medial-lateral direction, or a combination thereof. In some cases, an array of pressure sensors are integrated into the chest-mounted apparatus and utilized to detect the position of the compression.

In some aspects, feedback can be provided to a user or a device based on the position of the compression. For instance, the feedback can be generated in response to determining that the detected position of a previous compression is outside of a target range corresponding to an optimal chest compression position. In some cases, the feedback causes correction of the chest compression position, such that future chest compressions are administered within the target range.

According to some examples, the chest-mounted apparatus has additional features. For example, the chest-mounted apparatus may have a nonslip surface that further enhances chest compression efficacy. In some cases, additional sensors electrodes, for example electrocardiogram (ECG) electrodes are integrated into the chest-mounted apparatus. For instance, the chest-mounted apparatus further improves the placement of the electrodes on the chest of the subject for monitoring or electrotherapy purposes.

Various implementations of the present disclosure provide improvements to the technical field of emergency medical care. By enabling the detection and tracking of the position of chest compressions applied to a subject, various implementations described herein can be used to optimize or correct inadequate chest compression treatments. Accordingly, the condition of the subject can be improved. Moreover, various feedback described herein for optimizing chest compression position can enable untrained users (e.g., bystanders) to provide high-quality chest compression treatments. For instance, some implementations described herein could enable the use of public access chest compression devices that can be operated by anyone in the vicinity of a sudden medical emergency.

Implementations of the present disclosure will now be described with reference to the accompanying figures.

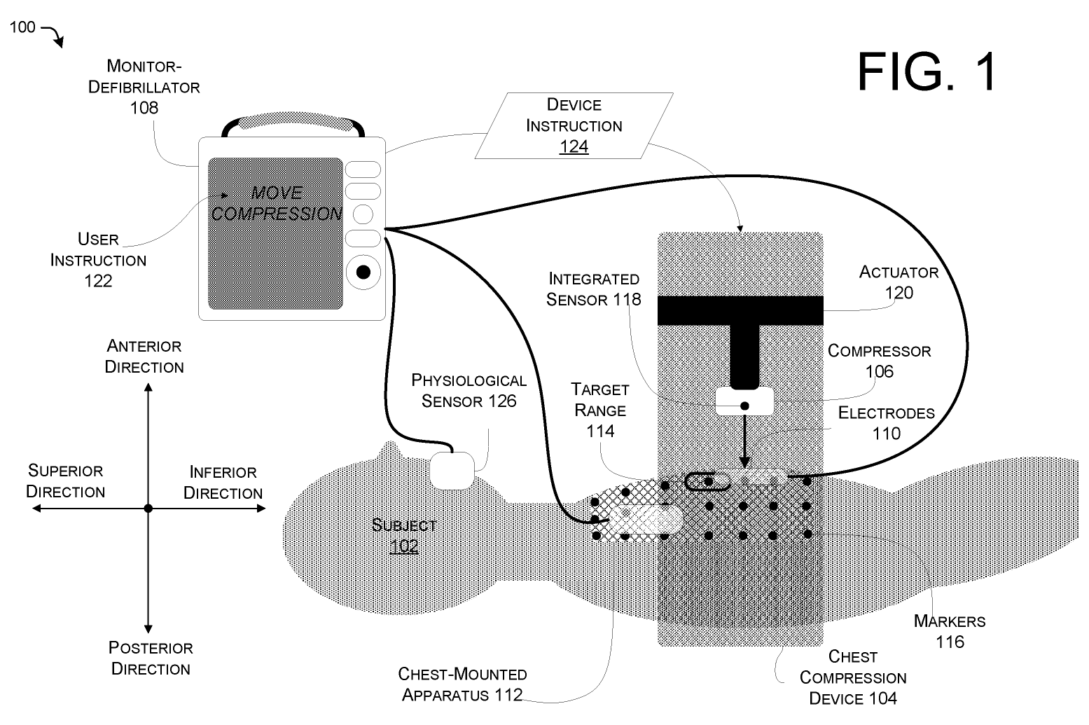

FIG. 1 illustrates an example environment 100 for tracking chest compression position. The environment 100, for instance, includes a rescue scene in which a subject 102 is experiencing a medical emergency. For example, the subject 102 may have suddenly lost consciousness within the environment 100. In some cases, the subject 102 is in cardiac arrest. The heart of the subject 102, for instance, may be at least temporarily unable to spontaneously circulate blood throughout the body of the subject 102.

In various cases, the environment 100 is in a clinical setting, such as a hospital. In some examples, the environment 100 is in a non-clinical, public setting such as an airport terminal, school, office building, or an outdoor environment. FIG. 1 illustrates anatomic directions relative to the position of the subject 102. For instance, a superior direction may extend from the feet to toward the head of the subject 102, an inferior direction may extend from the head toward the feet of the subject 102, an anterior direction may extend from a back to a front of the subject 102, and a posterior direction may extend from a front to a back of the subject 102. The term “anterior- posterior direction” may refer to the anterior direction or the posterior direction. The term “superior-inferior direction” may refer to the superior direction or the inferior direction.

A chest compression device 104 is an apparatus configured to administer chest compressions to the subject 102. The chest compressions, for instance, may at least temporarily induce blood flow in the body of the subject 102 while the heart of the subject 102 is unable to adequately and/or spontaneously circulate blood in the body. The induction of blood flow may deliver some oxygen to various tissues within the body of the subject 102. Accordingly, chest compressions may prevent hypoxic injury during a period of time in which the subject 102 lacks spontaneous circulation. In various cases, chest compressions can prevent damage to the brain, other vital organs, and other tissues within the body of the subject 102 during the medical emergency.

The chest compression device 104 includes a compressor 106 configured to compress the chest of the subject 102. In some cases, the compressor 106 includes a plunger that is configured to be periodically lowered onto the body of the subject 102 to administer compressions. Moreover, in some cases, the plunger is configured to pull up on the skin of the subject 102 between compressions, an act referred to herein as “active decompression.” In some examples, the compressor 106 is a band that is disposed around the body of the subject 102. For example, the band can administer compressions by being periodically tightened around the body of the subject 102.

In various implementations, a monitor-defibrillator 108 is also present within the environment 100. The monitor-defibrillator 108 is configured to monitor the subject 102 and/or to administer a treatment to the subject 102. In various cases, the monitor-defibrillator 108 is configured to provide feedback about a condition of the subject 102 to one or more users (e.g., trained rescuers or untrained bystanders). In particular examples, the monitor-defibrillator 108 is configured to provide feedback about a treatment (e.g., the chest compressions) administered to the subject 102.

In various cases, the monitor-defibrillator 108 is electrically coupled to electrodes 110 disposed on the chest of the subject 102. In various cases, the electrodes 110 are disposed on the chest of the subject 102 at anterior and lateral positions. The electrodes 110 include electrocardiogram (ECG) electrodes, electrotherapy (e.g., defibrillation) electrodes, or a combination thereof. In some examples, the electrodes 110 are adhered to the skin of the subject 102. In various cases, the monitor-defibrillator 108 is configured to detect an ECG of the subject 102 by detecting, at the electrodes 110, an electrical signal output by the heart of the subject 102. In some cases, the monitor-defibrillator 108 is configured to detect a cardiac arrhythmia of the subject 102 by analyzing the ECG. The monitor-defibrillator 108 may be configured to recommend, or initiate, administration of an electrotherapy to the subject 102 based on the detected cardiac arrhythmia. For instance, if the monitor-defibrillator 108 determines that the ECG of the subject 102 is indicative of ventricular fibrillation (VF) or ventricular tachycardia (VT), the monitor-defibrillator 108 may recommend, or prepare for, discharging an electrical shock to the electrodes 110. In various cases, the monitor-defibrillator 108 is configured to administer the electrotherapy to the subject 102 by discharging the electrical shock to the electrodes 110. While the monitor-defibrillator 108 and the chest compression device 104 are shown as separate devices, in some aspects they may be combined partially or entirely into a single device. For example, ECG electrodes similar to electrodes 110 may be incorporated into the chest-compression device 104.

While both the chest compression device 104 and the monitor-defibrillator 108 are capable of providing important context into the condition of the subject 102 and are also capable of providing vital treatments to the subject 102, the efficacy of both devices can be highly dependent on the expertise of the operator. For example, a user unfamiliar with operating the chest compression device 104 may incorrectly position the chest compression device 104 with respect to the body of the subject 102. For example, the chest compression device 104 may be a public access device, and the user may be an untrained bystander present in the environment 100 when the subject 102 experiences the medical emergency. Incorrectly positioning the chest compression device 104 may result in the compressor 106 compressing an incorrect position on the body of the subject 102. In various cases, applying compressions to the incorrect position can result in harm to the body of the subject 102. In various cases, applying compressions to the incorrect position can result in an inadequate flow of blood through the body of the subject 102, which can lead to a hypoxic injury to the subject 102. In various cases, incorrectly positioning the chest compression device 104 can cause serious harm to the subject 102, such as rib fractures, internal organ contusions, lacerated liver, and the like.

In some cases, the subject 102 can be harmed if the electrodes 110 are incorrectly positioned on the body of the subject 102. For instance, a user unfamiliar with operating the monitor-defibrillator 108 may position the electrodes 110 at incorrect positions on the body of the subject 102. If the electrodes 110 are incorrectly positioned, the monitor-defibrillator 108 may be unable to detect an accurate ECG of the subject 102, which can prevent the monitor-defibrillator 108 from accurately identifying whether the subject 102 has a treatable cardiac arrhythmia. Further, incorrectly positioning the electrodes 110 can prevent the monitor-defibrillator 108 from effectively treating the subject 102 with an electrotherapy. For instance, if the monitor-defibrillator 108 outputs an electrical shock to the electrodes 110, and an electrical path between the electrodes 110 minimally intersects the heart of the subject 102, the electrical shock may be unable to defibrillate the subject 102.

Other types of operational errors may result in improperly positioning the chest compression device 104 and electrodes 110. For instance, if the subject 102 is experiencing an acute medical emergency, the user of the devices may be experiencing immense stress and may be distracted by other facets of monitoring and treating the subject 102. In some cases, the user is administering assisted ventilation to the subject 102 using manual rescue breaths, and it may be difficult for the user to administer adequate ventilation while carefully positioning the chest compression device and electrodes 110. In some examples, the subject 102 is being transported to a clinical environment, such as in an ambulance, and the position of the chest compression device 104 with respect to the body of the subject 102 may shift over time.

Further, in some cases, the subject 102 may have unique physiology that makes it difficult to predict the appropriate placement of the chest compression device 104 and/or electrodes 110. In some examples, the subject 102 has dextrocardia, in which the heart of the subject 102 is located on the right side of the chest of the subject 102 rather than the left side of the chest of the subject 102. For instance, if the electrodes 110 are placed on the chest of the subject 102 with the assumption that the heart of the subject 102 is located on the left side of the chest, the electrodes 110 may be unable to accurately capture the ECG of the subject 102 and/or to administer an effective electrotherapy to the heart of the subject 102. The subject 102 may have one or more physiological characteristics that prevents accurate prediction of the appropriate placement of the chest compression device 104 or of the electrodes 110, even by a highly experienced and undistracted user.

Various implementations of the present disclosure address these and other problems using a chest-mounted apparatus 112. The chest-mounted apparatus 112, in various cases, is configured to be placed on the chest of the subject 102. The chest-mounted apparatus 112 is configured to be disposed across a plane that is normal to an anterior-posterior direction. For example, the chest mounted apparatus 112 may be large enough to sufficiently cover an area of the chest of the subject 102 that extends between skin covering a clavicle of the subject 102, skin covering one or more ribs of the subject 102, skin covering at least a portion of the abdomen of the subject 102, skin adjacent to one or more armpits of the subject 102, or any combination thereof. In some cases, the chest-mounted apparatus 112 is symmetric, with respect to a midline of the subject 102 after placement on the chest of the subject 102. The skin-mounted apparatus 112 may include one or more visual markers that enhances manual placement of the skin- mounted apparatus 112 on the chest of the subject 102. For instance, the skin-mounted apparatus 112 may include one or more visual markers indicating a left side of the skin-mounted apparatus 112, a center line of the chest-mounted apparatus 112, a right side of the skin-mounted apparatus 112, one or more relative locations of physiological markers (e.g., nipples, shoulder, collarbone, neck, etc.) of the subject 102 relative to the skin-mounted apparatus 112 after placement, or any combination thereof. In some cases, the chest-mounted apparatus 112 includes an adhesive configured to adhere the chest-mounted apparatus 112 to the skin of the subject 102. At least a portion of the adhesive may be biocompatible and/or electrically conductive, for instance.

The chest-mounted apparatus may be configured to conform to an external surface of the chest of the subject 102. For instance, the chest-mounted apparatus includes a flexible and/or elastic material. The chest-mounted apparatus, for example, includes a flexible and/or elastic housing that at least partially encloses electronic components of the chest-mounted apparatus 112. In particular examples, an exterior surface of the housing of the chest-mounted apparatus 112 has a relatively high coefficient of friction (e.g., 0.5 or greater), which may facilitate an interface between the compressor 106 and the chest-mounted apparatus 112. The housing of the chest-mounted apparatus 112, for instance, may enhance active decompression administered by the compressor 106 and prevent slippage between the compressor 106 and the chest-mounted apparatus 112.

In various cases, the chest-mounted apparatus 112 is configured to facilitate an effective placement of the compressor 106 on the chest of the subject 102. In various cases, the chest-mounted apparatus 112 includes a target range 114, which may represent an area corresponding to an average effective placement of the compressor 106. According to some cases, the target range 114 is predetermined. In some examples, when the chest-mounted apparatus 112 is disposed on the chest of the subject 102, the target range 114 overlaps a sternum (e.g., xiphoid process) of the subject 102, a midline of the subject 102, a line extending between the nipples of the subject 102, or any combination thereof. An indication of the target range 114, in some instances, is printed on an exterior of the chest-mounted apparatus 112. Accordingly, the user may position the chest compression device 104 by manually placing the compressor 106 at a position that is aligned with the target range 114.

In some cases, the target range 114 includes a fastener configured to physically couple to the compressor 106 to the target range 114 of the chest-mounted apparatus 112. The fastener, for instance, facilitates active decompression administered by the compressor 106 and may further prevent slippage between the compressor 106 and the chest-mounted apparatus 112. For instance, the compressor 106 may include a suction cup configured to conform to a fastening material disposed in the target range 114. The fastening material, for instance, includes a foam, gel, viscoelastic material, rheopectic material, or the like, which is configured to conform to the shape of the chest of the subject 102 but solidify when a pressure is applied, such as by the compressor 106 during chest compressions. In some cases, the compressor 106 and/or the target range 114 includes an adhesive. In some examples, the compressor 106 and/or the target range 114 includes a magnetic material (e.g., a ferromagnetic material) configured to bind the compressor 106 to the target range 114. In some cases, the target range 114 includes a mechanical fastener, such as a quick-connect fastener, a screw, and/or a release mechanism. The fastener, for instance, may have a flat profile on the surface of the chest-mounted apparatus 112. In some cases, the fastener is recessed into a thickness of the chest-mounted apparatus 112 and/or a recessed portion of the compressor 106 is configured to be removably connected to the fastener.

According to some examples, the chest-mounted apparatus 112 is configured to detect a position of a compression applied by the compressor 106. The chest-mounted apparatus 112 may include multiple markers 116 integrated with the chest-mounted apparatus 112. The markers 116, in various implementations, are spaced apart in a superior-inferior direction and/or a medial-lateral direction (e.g., a direction normal to a plane that includes the superior-inferior direction and the anterior-posterior direction). In some examples, an array of the markers 116 is distributed across the chest-mounted apparatus 112. For instance, different markers 116 may overlap different positions on the chest of the subject 102 when the chest-mounted apparatus 112 is disposed on the chest of the subject 102.

In some cases, the markers 116 include sensors configured to detect the compression. For instance, the sensors may include one or more pressure sensors. Examples of pressure sensors include, for instance, capacitive sensors, resistive sensors, piezoelectric sensors, triboelectric sensors, or a combination thereof. In various cases, the chest-mounted apparatus 112 includes a circuit configured to determine the position of the compression based on the one or more sensors among the markers 116 that detects the compression. The circuit, for instance, may generate an indication of the position of the compression based on at least one signal generated by the one or more sensors in response to being compressed by the compression. For example, the chest-mounted apparatus 112 may include an active device configured to detect the position of the compression. In various implementations, the chest-mounted apparatus 112 is configured to detect a position of the compression relative to the target range 114. For example, the chest-mounted apparatus 112 may determine whether the position of the compression is within the target range 114 or outside of the target range 114.

In various implementations, the chest-mounted apparatus 112 is configured to output an indication of the detected position of the compression. For example, the chest-mounted apparatus 112 may be communicatively coupled with the chest compression device 104, such as via at least one wired interface and/or at least one wireless interface. The chest-mounted apparatus 112, in some cases, is configured to output a signal indicating the detected position of the compression on the chest-mounted apparatus 112.

According to some examples, the monitor-defibrillator 108 is communicatively coupled with the chest-mounted apparatus 112 and is configured to determine the position of the compression and/or whether the compression is within the target range 114 based on a signal from the chest-mounted apparatus 112. For example, the chest-mounted apparatus 112 may be communicatively coupled with the monitor-defibrillator 108 via at least one wired interface and/or at least one wireless interface. The chest-mounted apparatus 112, in some cases, is configured to output a signal indicating the detected position of the compression on the chest-mounted apparatus 112 to the monitor-defibrillator 108.

In some implementations, the monitor-defibrillator 108 is configured to provide feedback about the position of the compression. For instance, if the monitor-defibrillator 108 determines that the compression is outside of the target range 114, the monitor-defibrillator 108 may output a user instruction 122 indicating that the position of future compressions should be adjusted. The user instruction 122, in some cases, is visually presented on a display of the monitor-defibrillator 108. In some cases, the user instruction 122 is audibly output to a user. In some cases, the user instruction 122 may direct the user to move the chest compression device 104 and/or compressor 106 in a particular direction (e.g., in a superior direction, in an inferior direction, or the like) to bring future compressions into the target range 114.

In some cases, the monitor-defibrillator 108 outputs a device instruction 124 to the chest compression device 104. In some cases, the device instruction 124 includes an indication of the position of the compression, a relative position of the compression with respect to the target range 114, or a direction to move the compressor 106 (e.g., in the superior direction, in the inferior direction, etc.) to bring future compressions into the target range 114.

In some examples, the chest-mounted apparatus 112 includes one or more passive elements. For example, the markers 116 may include fiducial markers that are detected by an integrated sensor 118 in the chest compression device 104. The integrated sensor 118, for instance, is disposed within, or is adjacent to, the compressor 106 of the chest compression device 104. The integrated sensor 118 may be configured to detect a distance between the compressor 106 and one or more of the markers 116 in the chest-mounted apparatus 112. In some implementations, the integrated sensor 118 includes an ultrasound transducer, an infrared distance sensor, a light detection and ranging (LIDAR) sensor, a time-of-flight sensor, or any combination thereof. For example, the integrated sensor 118 is configured to emit a signal (e.g., an ultrasound signal, a light signal, an electromagnetic signal, or the like), and detect the reflection of the signal from one or more of the fiducial markers. According to some examples, the integrated sensor 118 is configured to emit the signal periodically, such as at each compression administered by the compressor 106, between compressions administered by the compressor 106, or a combination thereof. In various cases, the markers 116 include a metal, an antenna, or any other material configured to reflect the signal emitted by the integrated sensor 118. The distance between the compressor 106 and one or more of the markers 116 may be determined by analyzing a time-of-flight between the emission of the signal and the receipt of the reflection of the signal by the integrated sensor 118. In some examples, each of the markers 116 has a distinct shape resulting in a distinct spectral signature of the reflection. For instance, the integrated sensor 118 and/or the chest-mounted apparatus 112 may be configured to detect which of the markers 116 the reflection was emitted from based on the spectral signature of the reflection. According to some cases, the integrated sensor 118 receives reflections from multiple markers 116, thereby enabling the integrated sensor 118 and/or the chest-mounted apparatus 112 to triangulate the position of the compressor 106 with respect to the chest-mounted apparatus 112.

Although not specifically illustrated in FIG. 1, in some implementations, the markers 116 or the integrated sensor 118 are integrated with a backboard configured to be positioned underneath the back of the subject 102. For instance, the backboard may be configured to detect a relative position of the backboard with respect to the chest-mounted apparatus 112 (and, thereby, determine the relative position of the backboard with respect to the body of the subject 102) by detecting the distances between the integrated sensor 118 and the markers 116. In some cases, the backboard includes an indicator that communicates, to a user, whether the backboard is aligned ideally with respect to the subject 102. In various cases, the backboard is positioned under the subject 102 before or after the chest compression device 104 is positioned over and/or around the subject 102.

In some instances, the integrated sensor 118 may detect whether the compressor 106 makes contact with one or more markers 116 within the target range 114 of the chest-mounted apparatus 112. For instance, the integrated sensor 118 may include an electrical circuit that generates, or modifies, an electrical signal within the electrical circuit when the compressor 106 is in contact with one or more of the markers 116. In some cases, the chest compression device 104 is configured to detect the position of the compression on the chest-mounted apparatus 112 based on the position of the compressor 106 detected by the integrated sensor 118.

In various cases, the chest compression device 104 is configured to automatically adjust the position of future compressions based on the determined position of the compression and/or based on the device instruction 124. For instance, the chest compression device 104 includes an actuator 120 configured to reposition the compressor 106 along an axis including a superior-inferior direction, or to reposition the compressor 106 along an axis normal to a plane including a superior-inferior direction and an anterior-posterior direction. For instance, the actuator 120 is configured to reposition the compressor 106 along a direction that is parallel to the upper surface of the chest-mounted apparatus 112. In various cases, the chest compression device 104 is configured to reposition, based on the signal from the chest-mounted apparatus, the compressor 106 to cause the position of future compressions to be within the target range 114.

In some examples, the target range 114 is determined based on a physiological response of the subject 102 to one or more treatments. In some cases, the target range 114 may be determined specifically for the subject 102, may be adjusted over time, or the like. For example, the monitor-defibrillator 108 may be configured to determine the target range 114 based on the determined position of the compression applied by the compressor 106 and the physiological response of the subject 102 to the compression.

In various cases, the monitor-defibrillator 108 includes, or is communicatively coupled with, a physiological sensor 126. The physiological sensor 126 is configured to detect a physiological parameter of the subject 102 that is indicative of blood circulation. Examples of physiological parameters include, for instance, blood pressure, blood flow (e.g., volumetric blood flow through one or more blood vessels, blood velocity in one or more blood vessels, blood flow rate, etc.), blood oxygenation (e.g., cerebral oxygenation, pulse oxygenation (SpO₂), regional oxygenation, plethysmograph, etc.), an airway parameter (e.g., a partial pressure of CO₂ in an airway of the subject 102, an end-tidal CO₂ (EtCO₂), a partial pressure of O₂ in the airway, a capnograph, etc.), a temperature, a transthoracic impedance, a pulse rate, or any combination thereof. For example, the physiological sensor 126 includes an invasive blood pressure sensor, a noninvasive blood pressure sensor (e.g., a blood pressure cuff), a flow sensor (e.g., an ultrasound transducer configured to detect a blood velocity using Doppler-based techniques), an oximeter, a gas sensor (e.g., a non-dispersive infrared (NDIR) CO₂ sensor), a thermometer, electrodes, or any combination thereof. The monitor-defibrillator 108, for instance, is configured to detect whether previously applied chest compressions are effective by comparing the physiological parameter to a threshold range. For instance, if the EtCO₂ of the subject 102 is below a threshold, the monitor-defibrillator 108 may infer that the compressions were ineffective. Upon determining that the compressions were ineffective, the monitor-defibrillator 108 may determine that the position of the compression was outside of the target range 114. In some cases, the monitor-defibrillator 108 is configured to determine that the physiological parameter of the subject 102 has entered the threshold range after the compressor 106 is repositioned, indicating that the position of the compressions is within the target range 114.

In some cases, the monitor-defibrillator 108 is configured to compare the physiological parameter of the subject 102 while the subject is receiving chest compressions at different positions. For example, the chest-mounted apparatus 112 may report the position of first compressions applied to the chest of the subject 102 and may also report the position of second compressions applied to the chest of the subject 102. In various cases, the monitor-defibrillator 108 is configured to compare a measurement of the physiological parameter when the subject 102 is receiving the first compressions and when the subject 102 is receiving the second compressions. Based on the comparison, the monitor-defibrillator 108 may be configured to determine whether the position of the first compressions is in the target range 114, or whether the position of the second compressions is in the target range 114. For example, of a blood oxygenation when the subject 102 is receiving the first compressions is greater than a blood oxygenation when the subject 102 is receiving the second compressions, the monitor-defibrillator 108 may determine that the position of the first compressions is in the target range 114, that the position of the second compressions is outside of the target range 114, that the position of the first compressions is closer to the target range 114 than the position of the second compressions, or a combination thereof. In an alternate example, if a physiological parameter (e.g., EtCO₂) of the subject 102 detected when the chest compressions are applied at a first position on the chest of the subject 102 is below a threshold, the monitor-defibrillator 108 may cause subsequent compressions to be applied at a second position on the chest of the subject 102, wherein the second position is to the left and/or inferior to the first position. For instance, the monitor-defibrillator 108 may cause the compressor 106 to be repositioned from the first position to the second position. In some implementations, changing the chest compression position can prevent the left ventricle outflow tract of the subject 102 from being compressed. Accordingly, the flow of blood ejected from the left ventricle can be enhanced. In various cases, the monitor-defibrillator 108 is configured to generate the user instruction 122 and/or the device instruction 124 based on the determined target range 114.

Although FIG. 1 has been described with reference to chest compressions applied by the chest compression device 104, implementations are not so limited. Using similar techniques to those described above, the chest-mounted apparatus 112 and/or monitor-defibrillator 108 may be configured to detect the position of compressions manually applied by a rescuer to the chest of the subject 102. For instance, the hands of the rescuer may be substituted for the compressor 106 in various implementations described herein. In some cases, the manual chest compressions are applied to the chest-mounted apparatus 112 and/or the integrated sensor 118. In some cases, the integrated sensor 118 is integrated, or is configured to detect, a glove, a ring, a watch, a patch, or a puck attached to the hands of the rescuer providing manual chest compressions. Further, the monitor-defibrillator 108 may be configured to provide feedback about the manual chest compressions to the rescuer via the user instruction 122.

In some implementations, the electrodes 110 are integrated into the chest-mounted apparatus 112. In some examples, the chest-mounted apparatus 112 includes a band that extends between the electrodes 110. Cables configured to electrically and/or communicatively connect the electrodes 110 to the monitor-defibrillator 108 may also be part of the chest-mounted apparatus 112. In various cases, connectors are disposed on the cables, which are configured to be removably coupled with one or more ports of the monitor-defibrillator 108. For example, the electrodes 110 may be disposed in a lower layer of the chest-mounted apparatus 112 than the markers 116, such that the electrodes 110 are closer to the skin of the subject 102 when the chest-mounted apparatus 112 is disposed on the chest of the subject 102. In some cases, it may be easier to place the entire chest-mounted apparatus 112 on the chest of the subject 102 than the individual electrodes 110. Thus, integrating the electrodes 110 into the chest-mounted apparatus 112 may prevent a user from placing the electrodes 110 in incorrect positions on the body of the subject 102. In some cases, various components (e.g., the electrodes 110, the sensor 118, etc.) of the chest-mounted apparatus 112 are included in one or more flexible electrical circuits included in the chest-mounted apparatus 112.

In some cases, multiple pairs of electrode 110 are integrated into the chest-mounted apparatus 112. The monitor-defibrillator 108, for instance, is configured to selectively activate a pair of the electrodes 110 (among the multiple pairs) for monitoring and/or treatment based on a relative size of the subject 102. For example, if the subject 102 is relatively small, the monitor-defibrillator 108 is configured to activate a pair of the electrodes 110 that is adjacent to a center line of the chest-mounted apparatus 112 (e.g., a proximal pair of the electrodes 110). In contrast, if the subject 102 is relatively large, the monitor-defibrillator 108 is configured to activate a pair of the electrodes 110 that is disposed on an outside edge of the chest-mounted apparatus 112 (e.g., a distal pair of the electrodes 110). The size of the subject 102, for instance, can be input into the monitor-defibrillator 108 by a user.

Although various techniques described herein are illustrated as being performed by the chest compression device 104 and/or the monitor-defibrillator 108, implementations of the present disclosure are not so limited. Unless otherwise specified, any of the techniques described herein can be performed by other types of medical devices (e.g., automated external defibrillators (AEDs)), generic computing devices, or the like.

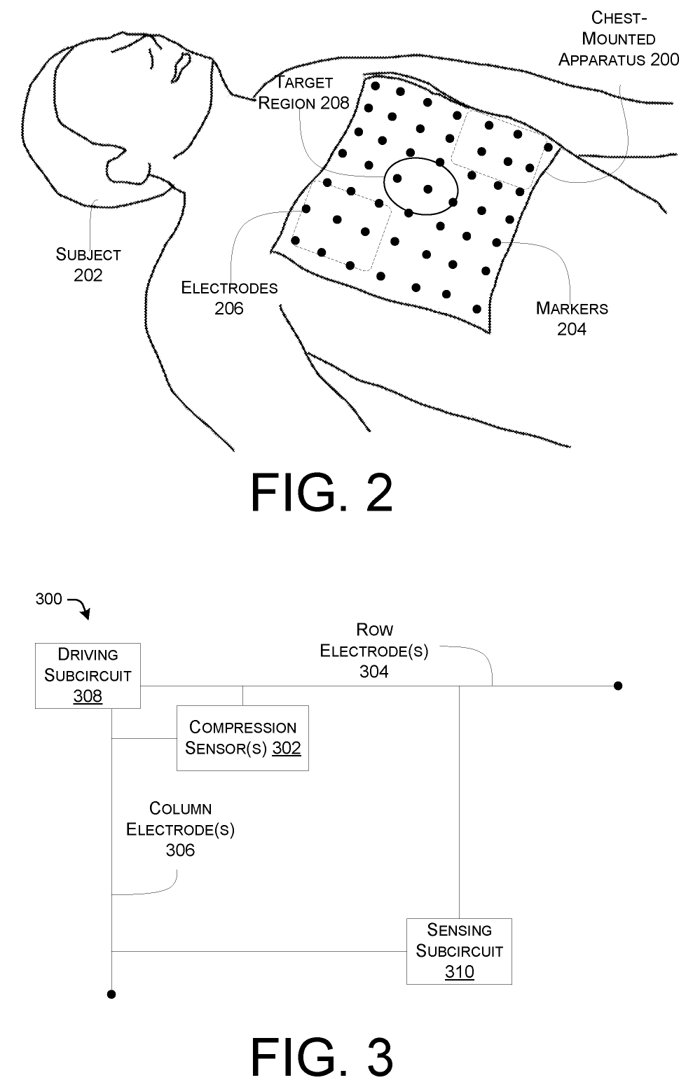

FIG. 2 illustrates an example of a chest-mounted apparatus 200 disposed on the chest of a subject 202. For example, the chest-mounted apparatus 200 may correspond to the chest-mounted apparatus 112 described above with reference to FIG. 1.

In various cases, the chest-mounted apparatus 200 is configured to substantially cover the chest of the subject 202 when applied to the subject 202. The chest-mounted apparatus 200, in various cases, includes a flexible and/or elastic housing configured to conform to an exterior shape of the chest of the subject 202. The chest-mounted apparatus 200, in some cases, includes an adhesive layer configured to adhere the housing of the chest-mounted apparatus 200 to the skin of the subject 202. In some cases, the adhesive is electrically conductive. For instance, the adhesive may be a water-based adhesive including one or more electrolytes.

In various cases, the chest-mounted apparatus 200 includes an array of markers 204 (e.g., sensors and/or fiducial markers) configured to enable the determination of a position of one or more compressions applied to the chest-mounted apparatus 200 and to the chest of the subject 202. The chest-mounted apparatus 200 additionally includes electrodes 206 configured to be disposed adjacent to the skin of the chest of the subject 202. For instance, the electrodes 206 may be disposed between the markers 204 and an adhesive layer (e.g., disposed on the skin of the subject 202) of the chest-mounted apparatus 200. In some cases, an electrically inductive material is disposed between the electrodes 206 and the markers 204, which may protect a circuit including the markers 204 from an electrical signal discharged by the electrodes 206 to the subject 202.

The chest-mounted apparatus 200, for instance, includes a target region 208. When the chest-mounted apparatus 200 is disposed on the chest of the subject 202, the target region 208 may correspond to a position on the chest of the subject 202 that is an optimal chest compression position. In some cases, the target region 208 is printed on an exterior of the housing of the chest-mounted apparatus 200. In various implementations, the target region 208 is calculated based on a response of the subject 202 to one or more treatments (e.g., chest compressions).

FIG. 3 illustrates an example circuit 300 for detecting a position of a compression applied to an apparatus. For example, the circuit 300 may be configured to detect the position of a compression applied to a chest-mounted apparatus, such as the chest-mounted apparatus 112 and/or the chest-mounted apparatus 200 described above. In some cases, the circuit 300 is integrated within the chest-mounted apparatus.

The circuit 300 includes one or more compression sensors 302 disposed at one or more predetermined positions within the chest-mounted apparatus. For example, the compression sensor(s) 302 include an array of sensors distributed throughout the chest-mounted apparatus. In some examples, the compression sensor(s) 302 are respectively electrically coupled with one or more row electrodes 304 and one or more column electrodes 306. For instance, multiple compression sensor(s) 302 are connected to the same row electrode 304 and multiple compression sensor(s) 302 are connected to the same column electrode 306. However, in various cases, each compression sensor(s) 302 is connected to a different combination of the row electrode(s) 304 and the column electrode(s) 306. The compression sensor(s) 302, in various cases, include one or more capacitive sensors, one or more resistive sensors, one or more piezoelectric sensors, one or more triboelectric sensors, or any combination thereof.

A driving subcircuit 308 and a sensing subcircuit 310 may be electrically connected with the row electrode(s) 304 and the column electrode(s) 306. In some examples, the driving subcircuit 308 is configured to output one or more electrical signals to the row electrode(s) 304 and the column electrode(s) 306. For instance, the driving subcircuit 308 is configured to output a predetermined electrical current or voltage to the row electrode(s) 304 and the column electrode(s) 306. In some cases, the driving subcircuit 308 applies the electrical signal(s) to respective row electrode(s) 304 or column electrode(s) 306 at respective time intervals.

When a compression force is applied to one of the compression sensor(s) 302, the applied sensor may generate a change in the electrical signal in its connected row electrode 304 and in its connected column electrode 306. The sensing subcircuit 310, in various cases, is configured to detect the row electrode(s) 304 and column electrode(s) 306 reflecting a change in the electrical signal(s), which may be due to the activation of one or more of the compression sensor(s) 302. For example, a compression applied to one of the compression sensor(s) 302 may induce a change in a capacitance of a capacitor within the compression sensor 302, which may change a current and/or voltage applied to the capacitor. In some cases, a compression applied to one of the compression sensor(s) 302 may induce a change in a resistance of a resistor within the compression sensor 302, which may change a current and/or voltage applied to the resistor. Based on the electrical signal(s) output by the driving subcircuit 308, and the change in the electrical signal(s) detected by the sensing subcircuit 310, the compression sensor 302 to which the compression has been applied can be determined. Moreover, if the compression sensor 302 is in a fixed location within the chest-mounted apparatus, the position of the compression on the chest-mounted apparatus can be further determined.

The driving subcircuit 308 and the sensing subcircuit 310 may be separate circuit elements or may be combined into a single element. In various cases, the driving subcircuit 308 and/or the sensing subcircuit 310 include one or more integrated circuits (ICs), a processor, one or more analog-to-digital converters, or any combination thereof. The driving subcircuit 308 and the sensing subcircuit 310, for instance, may be powered by a power source, such as a battery, capacitor, or the like. In various cases, the chest-mounted apparatus, including the circuit 300, is a portable device. In some examples, the circuit 300 further includes a transceiver configured to output an indication of the position of the compression to an external device.

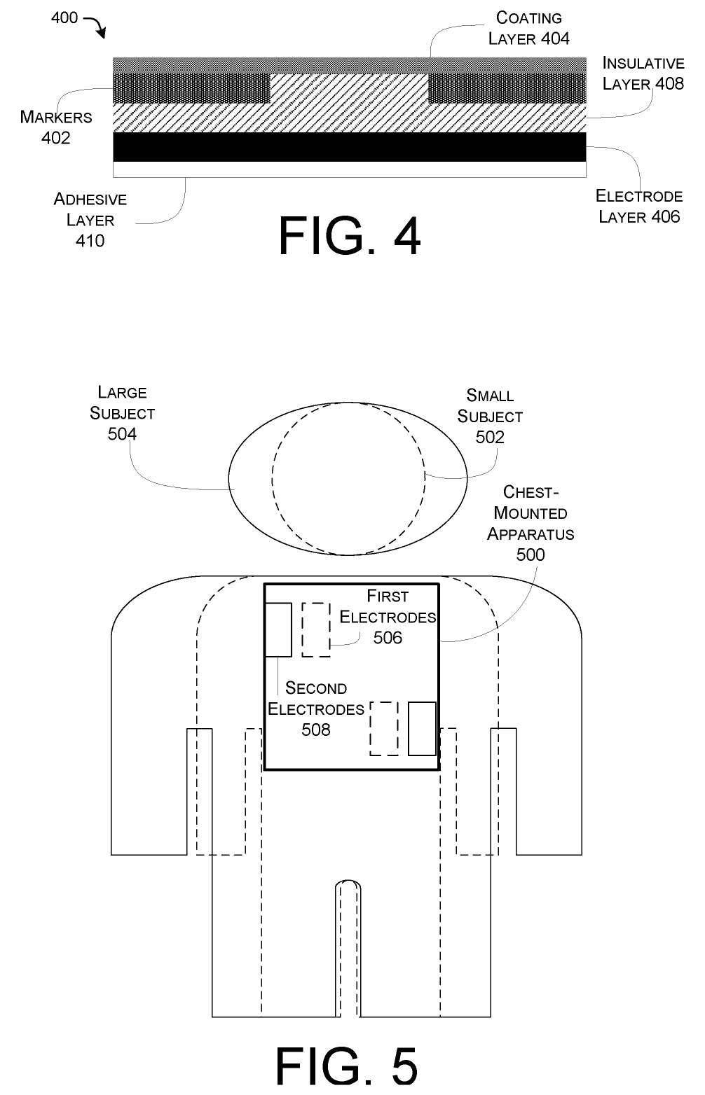

FIG. 4 illustrates an example cross-section of a portion of a chest-mounted apparatus 400. The chest-mounted apparatus 400, for instance, corresponds to the chest-mounted apparatus 112 and/or the chest-mounted apparatus 200 described above. In various cases, the chest-mounted apparatus 400 is flexible and/or elastic, and is configured to be disposed on the skin of a chest of a subject.

The chest-mounted apparatus 400 includes various markers 402 configured to be utilized to detect the position of one or more compressions applied to the chest-mounted apparatus 400. For example, the markers 402 include sensors and/or fiducial markers. The markers 402, in some cases, are part of a circuit disposed within the chest-mounted apparatus 400.

A coating layer 404 may be disposed on the markers 402. For instance, the markers 402 are configured to detect the position of a compression applied to the coating layer 404, such as by a compressor of a chest compression device or by hands of a rescuer. The coating layer 404 may be disposed on an upper surface of the chest-mounted apparatus 400. The coating layer 404, in various implementations, is configured to seal components of the chest-mounted apparatus 400 from an external environment. For instance, the coating layer 404 may prevent dust, moisture, and other environmental materials from touching the markers 402 and/or the circuit including the markers 402. In various cases, the coating layer 404 includes a flexible and/or elastic material. In some cases, the coating layer 404 includes a woven material. For example, the coating layer 404 may include a silicone, a polyester, a polyimide, polyether ether ketone (PEET), polyethylene terephthalate (PET), polystyrene, polyethylene, or any combination thereof. In various cases, a coefficient of friction of the coating layer 404 is greater than 0.1, 0.2, 0.3, 0.4, 0.5, or 0.6. For instance, a surface of the coating layer 404 may be etched or cast in a shape that increases the coefficient of friction compared to a smooth surface.

In various implementations, an electrode layer 406 is additionally disposed within the chest-mounted apparatus 400. For example, the electrode layer 406 includes one or more ECG electrodes, one or more electrotherapy (e.g., defibrillation) electrodes, or any combination thereof. The electrode layer 406 may include a flexible film. For instance, the flexible film includes silver and/or silver chloride, tin, or any combination thereof. In various cases, the electrode layer 406 is configured to be electrically coupled with a discharge circuit of a defibrillator. For example, the electrode layer 406 is configured to discharge an electrical shock (e.g., at an energy level of 200 Joules (J) or greater) provided by the discharge circuit.

To prevent the electrical shock from damaging the markers 402 and/or other circuit elements of the chest-mounted apparatus 400, the chest-mounted apparatus 400 may have an insulative layer 408 disposed between the markers 402 and the electrode layer 406. The insulative layer 408 may include an electrically insulative material, such as an insulative polymer. In various cases, the insulative layer 408 includes a polyester, a polyimide, PEET, PET, polystyrene, polyethylene, or any combination thereof. In some examples, the insulative layer 408 includes a polymer foam.

The electrode layer 406 is disposed between the insulative layer 408 and an adhesive layer 410. The adhesive layer 410, for instance, is configured to removably attach the chest-mounted apparatus 400 to skin of a subject. In some examples, the adhesive layer 410 includes a hydrogel containing electrolytes. In various cases, the adhesive layer 410 includes an acrylic adhesive.

FIG. 5 illustrates an example chest-mounted apparatus 500 with selectable electrodes based on subject size. For instance, the chest-mounted apparatus 500 may correspond to the chest-mounted apparatus 112, the chest-mounted apparatus 200, the chest-mounted apparatus 400, or any combination thereof.

In various cases, the chest-mounted apparatus 500 is configured to be used on a small subject 502 or a large subject 504. For example, the chest-mounted apparatus 500 may be packaged and/or designed for a single-use. In a sudden medical emergency, the chest-mounted apparatus 500 can assist with the monitoring and/or treatment of a patient regardless of whether they are the small subject 502 or the large subject 504. Due to the difference in physiology between the small subject 502 and the large subject 504, they may be associated with different ideal relative distances and placements of electrodes for monitoring (e.g., ECG) and/or treatment (e.g., electrotherapy).

To address this difference, for instance, the chest-mounted apparatus 500 includes multiple pairs of electrodes to be activated for different patient sizes. In the example of FIG. 5, the chest-mounted apparatus 500 includes first electrodes 506 and second electrodes 508. The first electrodes 506, for instance, are closer together on the chest-mounted apparatus 500 than the second electrodes 508. In various cases, the first electrodes 506 may be better for monitoring and/or treatment of the small subject 502, whereas the second electrodes 508 may be better for monitoring and/or treatment of the large subject 504. In various cases, a defibrillator (not illustrated) may selectively connect to the first electrodes 506 or the second electrodes 508 based on the size of the subject to which the chest-mounted apparatus 500 is applied.

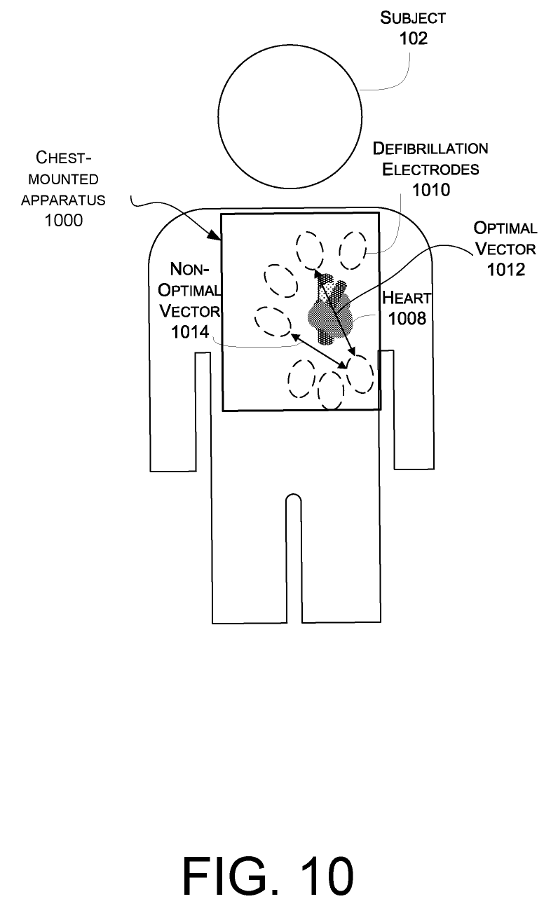

In some aspects, the electrodes may be disposed to define multiple shock vectors. In some cases, multiple electrodes are integrated into a single electrode pad (such as first electrode 506 and second electrode 508) that is disposed on the skin of the subject. While two electrodes/electrode pads are shown in FIG. 5 there may be more electrodes/electrode pads incorporated into the chest-mounted apparatus 500, as shown for example, in FIG. 10. The chest-mounted apparatus 500 outputs electrical signals along the multiple shock vectors and detects feedback based on the electrical signals. Based on the feedback, the chest-mounted apparatus may select one or more optimal vectors that are predicted to be optimal for a defibrillation therapy. In some cases, the chest-mounted apparatus recommends administration of one or more electrical shocks along the optimal vector(s). For instance, the chest-mounted apparatus recommends administration of a multi-shock therapy (e.g., a DSD therapy) including multiple electrical shocks along multiple optimal vectors.

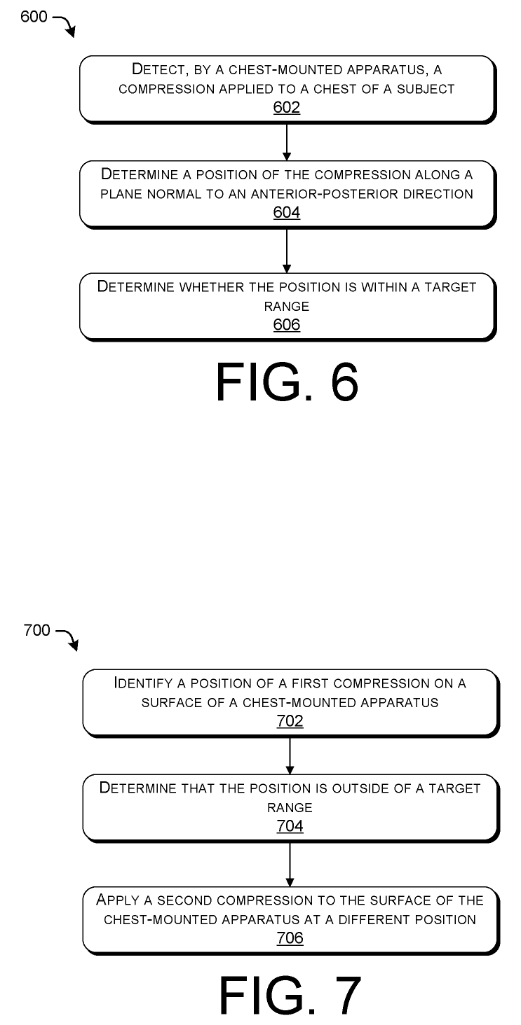

FIG. 6 illustrates an example process 600 for analyzing the position of chest compressions applied to a subject. The process 600 is performed by an entity, such as a monitor, a defibrillator, a monitor-defibrillator, an AED, a medical device, a chest compression device, a chest-mounted apparatus, a computing device, at least one processor, or any combination thereof.

At 602, the entity detects, by a chest-mounted apparatus, a compression applied to a chest of a subject. In various cases, the chest-mounted apparatus is configured to be disposed on the chest of the subject. For instance, the chest-mounted apparatus includes a flexible housing that is configured to be adhered to the skin on the chest of the subject by an adhesive. In some implementations, the housing includes a material (e.g., on a surface) that has a coefficient of friction that is 0.5 or greater. In various implementations, the chest-mounted apparatus further includes a circuit configured to detect the position of the compression. The compression, for instance, is applied by a compressor, a rescuer’s hands, or the like. In some cases, the circuit is communicatively coupled with a cable. The cable may be further attached to a connector configured to removably connect with a port of a device (e.g., a medical device, the entity, or the like). For instance, the chest-mounted apparatus transmits, via the cable, a communication signal indicating the detected compression to the device. Optionally, the chest-mounted apparatus further includes electrodes that can be electrically coupled to a device, thereby enabling the device to detect an electrical signal (e.g., indicative of an ECG) from and/or to output an electrical signal (e.g., an electrotherapy) to the subject. For instance, the device may administer an electrical shock to the electrodes integrated into the chest-mounted apparatus in response to detecting that the subject has an ECG indicative of VF. The electrical signal(s) can be transmitted via the cable, in some examples.

At 604, the entity determines a position of the compression along a plane normal to an anterior-posterior direction. The circuit, for instance, includes an array of sensors configured to detect the position along a plane that is normal to an anterior-posterior direction of the subject. The sensors, for instance, include at least one of capacitive sensors, resistive sensors, piezoelectric sensors, or triboelectric sensors. In various cases, the sensors include at least two sensors separated from each other in the chest-mounted apparatus along a superior-inferior direction, at least two sensors separated from each other in the chest-mounted apparatus along a medial lateral direction, or a combination thereof.

At 606, the entity determines whether the position is within a target range. In some cases, the chest-mounted apparatus includes a visual marker indicating the target range. The target range may be predetermined, or may be derived based on a physiological response of the subject to the compression. In various implementations, the entity generates feedback based on whether the position is within the target range. For instance, the entity generates a user instruction directing the user to maintain or adjust the position of future compressions to occur within the target range. In some examples, the entity generates a device instruction that causes the mechanical chest compression device to maintain or adjust the position of the future compressions to occur within the target range.

FIG. 7 illustrates an example process 700 for adjusting the position of chest compressions applied to a subject. The process 700 is performed by an entity, such as a monitor, a defibrillator, a monitor-defibrillator, an AED, a medical device, a chest compression device, a chest-mounted apparatus, a computing device, at least one processor, or any combination thereof.

At 702, the entity identifies a position of a first compression on a surface of a chest-mounted apparatus. In various cases, the chest-mounted apparatus is configured to be disposed on the chest of the subject. For instance, the chest-mounted apparatus includes a flexible housing that is configured to be adhered to the skin on the chest of the subject by an adhesive. In some implementations, the housing includes a material (e.g., on a surface) that has a coefficient of friction that is 0.5 or greater. In various implementations, the chest-mounted apparatus further includes a circuit configured to detect the position of the compression. In some examples, the chest-mounted apparatus is configured to be removably coupled with a compressor of the chest compression device. In some examples, the chest-mounted apparatus is configured to be removably coupled with hands of a user.

The compression, for instance, is applied by a compressor, a rescuer’s hands, or the like. In some cases, the circuit is communicatively coupled with a cable. The cable may be further attached to a connector configured to removably connect with a port of a device (e.g., a medical device, the entity, or the like). For instance, the chest-mounted apparatus transmits, via the cable, a communication signal indicating the detected compression to the device. In some cases, the chest-mounted apparatus includes a transceiver configured to transmit the communication signal via a wireless interface. Optionally, the chest-mounted apparatus further includes electrodes that can be electrically coupled to a device, thereby enabling the device to detect an electrical signal (e.g., indicative of an ECG) from and/or to output an electrical signal (e.g., an electrotherapy) to the subject. For instance, the device may administer an electrical shock to the electrodes integrated into the chest-mounted apparatus in response to detecting that the subject has an ECG indicative of VF. The electrical signal(s) can be transmitted via the cable, in some examples.

In some examples, the chest-mounted apparatus includes fiducial markers that can be detected by a sensor integrated with a chest compression device administering the compressions. For example, the sensor is integrated with the compressor of the chest compression device. In various cases, the sensor detects a distance between the sensor and one or more of the fiducial markers. At least one of the fiducial markers may be located within the target range. The entity may determine whether the compressor has applied the compression to the target range based on the detected distance between the sensor and the fiducial marker(s).

At 704, the entity determines that the position is outside of a target range (also referred to as a “target area”). In some cases, the chest-mounted apparatus includes a visual marker indicating the target range. The target range may be predetermined, or may be derived based on a physiological response of the subject to the compression. For example, the entity may detect, using a sensor, a physiological parameter of the subject indicative of blood circulation (e.g., a partial pressure of CO₂ or O₂ in an airway of the subject, a blood oxygenation of the subject, a blood flow rate of the subject, a blood pressure of the subject, etc.). If the physiological parameter detected after the compression, or during the compression, is outside of a threshold range (e.g., lower than a threshold or higher than a threshold), then the entity may infer that the compression is outside of the target range. In various implementations, the entity provides feedback about the relative position of the compression with respect to the target range, in order to facilitate the placement of future compressions within the target range. The feedback, in some cases, is in the form of a user instruction and/or a device instruction.

At 706, the entity applies a second compression to the surface of the chest-mounted apparatus at a different position. For instance, the entity may cause an actuator of the chest compression device to change the position of the compressor with respect to a plane normal to an anterior-posterior direction. The adjusted position, in various cases, is within the target range.

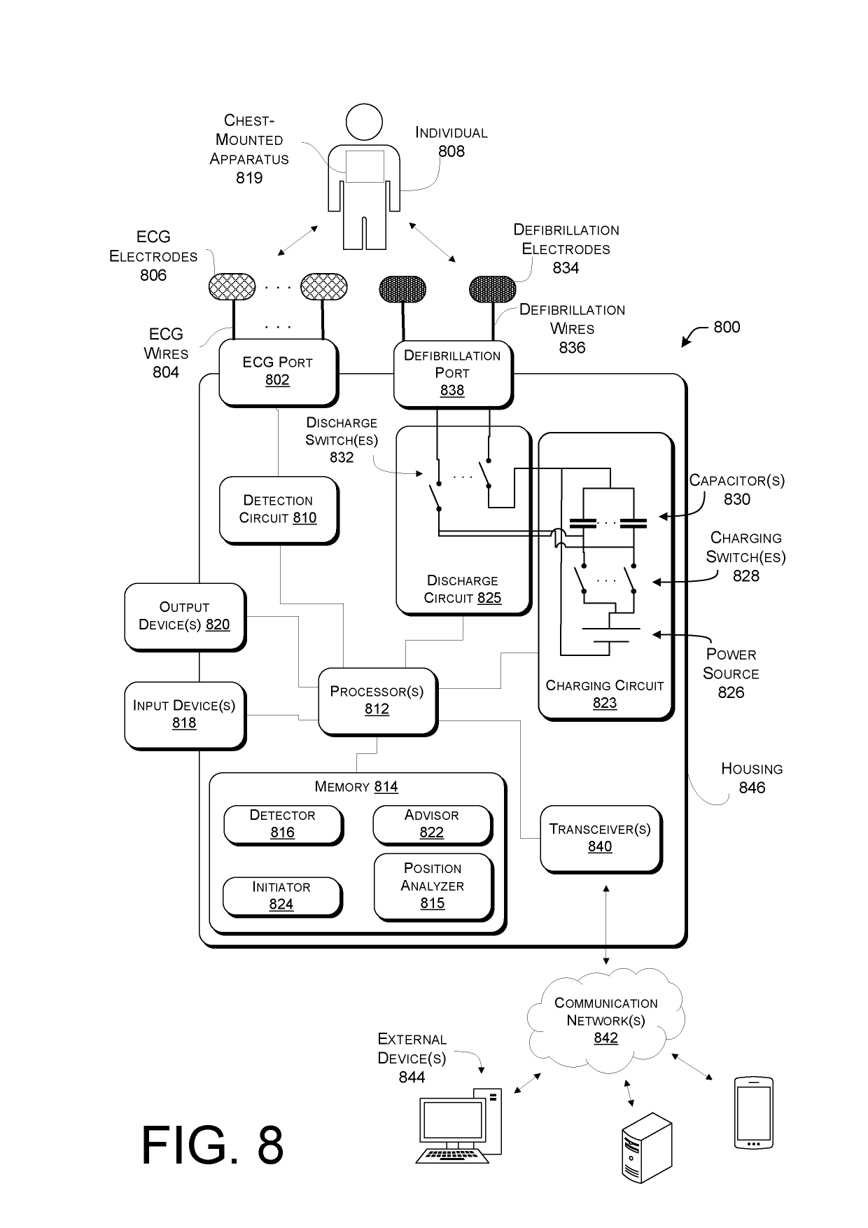

FIG. 8 illustrates an example of an external defibrillator 800 configured to perform various functions described herein. For example, the external defibrillator 800 is the monitor-defibrillator 108 described above with reference to FIG. 1. In some aspects the external defibrillator 800 may be combined with the chest mounted apparatus 819. In other aspects, they may be separate devices.

The external defibrillator 800 includes an electrocardiogram (ECG) port 802 connected to multiple ECG wires 804. In some cases, the ECG wires 804 are removeable from the ECG port 802. For instance, the ECG wires 804 are plugged into the ECG port 802 via connectors. The ECG wires 804 are connected to ECG electrodes 806, respectively. In various implementations, the ECG electrodes 806 are disposed on different locations on an individual 808. A detection circuit 810 is configured to detect relative voltages between the ECG electrodes 806. These voltages are indicative of the electrical activity of the heart of the individual 808.

In various implementations, the ECG electrodes 806 are in contact with the different locations on the skin of the individual 808. In some examples, a first one of the ECG electrodes 806 is placed on the skin between the heart and right arm of the individual 808, a second one of the ECG electrodes 806 is placed on the skin between the heart and left arm of the individual 808, and a third one of the ECG electrodes 806 is placed on the skin between the heart and a leg (either the left leg or the right leg) of the individual 808. In these examples, the detection circuit 810 is configured to measure the relative voltages between the first, second, and third ECG electrodes 806. Respective pairings of the ECG electrodes 806 are referred to as “leads,” and the voltages between the pairs of ECG electrodes 806 are known as “lead voltages.” In some examples, more than three ECG electrodes 806 are included, such that 5-lead or 12-lead ECG signals are detected by the detection circuit 810.

The detection circuit 810 includes at least one analog circuit, at least one digital circuit, or a combination thereof. The detection circuit 810 receives the analog electrical signals from the ECG electrodes 806, via the ECG port 802 and the ECG wires 804. In some cases, the detection circuit 810 includes one or more analog filters configured to filter noise and/or artifact from the electrical signals. The detection circuit 810 includes an analog-to-digital (ADC) in various examples. The detection circuit 810 generates a digital signal indicative of the analog electrical signals from the ECG electrodes 806. This digital signal can be referred to as an “ECG signal” or an “ECG.”

In some cases, the detection circuit 810 further detects an electrical impedance between at least one pair of the ECG electrodes 806. For example, the detection circuit 810 includes, or otherwise controls, a power source that applies a known voltage (or current) across a pair of the ECG electrodes 806 and detects a resultant current (or voltage) between the pair of the ECG electrodes 806. The impedance is generated based on the applied signal (voltage or current) and the resultant signal (current or voltage). In various cases, the impedance corresponds to respiration of the individual 808, chest compressions performed on the individual 808, and other physiological states of the individual 808. In various examples, the detection circuit 810 includes one or more analog filters configured to filter noise and/or artifact from the resultant signal. The detection circuit 810 generates a digital signal indicative of the impedance using an ADC. This digital signal can be referred to as an “impedance signal” or an “impedance.”

The detection circuit 810 provides the ECG signal and/or the impedance signal one or more processors 812 in the external defibrillator 800. In some implementations, the processor(s) 812 includes a central processing unit (CPU), a graphics processing unit (GPU), both CPU and GPU, or other processing unit or component known in the art.

The processor(s) 812 is operably connected to memory 814. In various implementations, the memory 814 is volatile (such as random access memory (RAM)), non-volatile (such as read only memory (ROM), flash memory, etc.) or some combination of the two. The memory 814 stores instructions that, when executed by the processor(s) 812, causes the processor(s) 812 to perform various operations. In various examples, the memory 814 stores methods, threads, processes, applications, objects, modules, any other sort of executable instruction, or a combination thereof. In some cases, the memory 814 stores files, databases, or a combination thereof. In some examples, the memory 814 includes, but is not limited to, RAM, ROM, electrically erasable programmable read-only memory (EEPROM), flash memory, or any other memory technology. In some examples, the memory 814 includes one or more of CD-ROMs, digital versatile discs (DVDs), content-addressable memory (CAM), or other optical storage, magnetic cassettes, magnetic tape, magnetic disk storage or other magnetic storage devices, or any other medium which can be used to store the desired information and which can be accessed by the processor(s) 812 and/or the external defibrillator 800. In some cases, the memory 814 at least temporarily stores the ECG signal and/or the impedance signal.

In various examples, the memory 814 includes a detector 816, which causes the processor(s) 812 to determine, based on the ECG signal and/or the impedance signal, whether the individual 808 is exhibiting a particular heart rhythm. For instance, the processor(s) 812 determines whether the individual 808 is experiencing a shockable rhythm that is treatable by defibrillation. Examples of shockable rhythms include ventricular fibrillation (VF) and ventricular tachycardia (VT). In some examples, the processor(s) 812 determines whether any of a variety of different rhythms (e.g., asystole, sinus rhythm, atrial fibrillation (AF), etc.) are present in the ECG signal.

The processor(s) 812 is operably connected to one or more input devices 818 and one or more output devices 820. Collectively, the input device(s) 818 and the output device(s) 820 function as an interface between a user and the defibrillator 800. The input device(s) 818 is configured to receive an input from a user and includes at least one of a keypad, a cursor control, a touch-sensitive display, a voice input device (e.g., a microphone), a haptic feedback device (e.g., a gyroscope), or any combination thereof. The output device(s) 820 includes at least one of a display, a speaker, a haptic output device, a printer, or any combination thereof. In various examples, the processor(s) 812 causes a display among the input device(s) 818 to visually output a waveform of the ECG signal and/or the impedance signal. In some implementations, the input device(s) 818 includes one or more touch sensors, the output device(s) 820 includes a display screen, and the touch sensor(s) are integrated with the display screen. Thus, in some cases, the external defibrillator 800 includes a touchscreen configured to receive user input signal(s) and visually output physiological parameters, such as the ECG signal and/or the impedance signal.

In various implementations, the input device(s) 818 further include, or are otherwise connected to, one or more sensors, such as physiological sensors. The physiological sensor(s), for instance, are configured to detect one or more physiological parameters of the individual 808. Examples of the physiological sensor(s) include a blood pressure sensor (e.g., a blood pressure cuff, invasive blood pressure sensor, or the like), an airway sensor (e.g., a sensor configured to detect a partial pressure of CO₂ and/or O₂ in an airway of the individual 808), a blood oxygenation sensor (e.g., a pulse oximeter, regional oxygenation sensor, or the like), a thermometer, a pulse sensor, a blood flow sensor (e.g., an ultrasound transducer configured to detect blood flow using Doppler-based techniques), an airway pressure sensor, or any combination thereof. The input device(s) 818, in some cases, includes one or more sensors configured to detect other characteristics of the individual 808. For example, the input device(s) 818 includes an accelerometer, gyroscope, microphone, or any combination thereof. In various implementations, the processor(s) 812 is configured to assess a condition of the individual 808 by analyzing data derived from signals detected by the input device(s) 818. According to some cases, the input device(s) 818 includes and/or are connected to a chest-mounted apparatus 819 configured to detect the application of a compression on the chest of the individual 808.

In some examples, the memory 814 includes an advisor 822, which, when executed by the processor(s) 812, causes the processor(s) 812 to generate advice and/or control the output device(s) 820 to output the advice to a user (e.g., a rescuer). In some examples, the processor(s) 812 provides, or causes the output device(s) 820 to provide, an instruction to perform CPR on the individual 808. In some cases, the processor(s) 812 evaluates, based on the ECG signal, the impedance signal, or other physiological parameters, CPR being performed on the individual 808 and causes the output device(s) 820 to provide feedback about the CPR in the instruction. According to some examples, the processor(s) 812, upon identifying that a shockable rhythm is present in the ECG signal, causes the output device(s) 820 to output an instruction and/or recommendation to administer a defibrillation shock to the individual 808.

In some implementations, the memory 814 further includes instructions for executing a position analyzer 815. For example, when the processor(s) 812 executes the position analyzer 815, the external defibrillator 800 may be configured to perform various operations described herein, such as determining the position of a chest compression applied to the individual 808 (e.g., based on a signal communicated by the chest-mounted apparatus 819), determining a target range of the chest-mounted apparatus 819, generating a user instruction based on the position of the chest compression and/or the target range, generating a device instruction based on the position of the chest compression and/or the target range, or any combination thereof.

The memory 814 also includes an initiator 824 which, when executed by the processor(s) 812, causes the processor(s) 812 to control other elements of the external defibrillator 800 in order to administer a defibrillation shock to the individual 808. In some examples, the processor(s) 812 executing the initiator 824 selectively causes the administration of the defibrillation shock based on determining that the individual 808 is exhibiting the shockable rhythm and/or based on an input from a user (received, e.g., by the input device(s) 818. In some cases, the processor(s) 812 causes the defibrillation shock to be output at a particular time, which is determined by the processor(s) 812 based on the ECG signal and/or the impedance signal.

The processor(s) 812 is operably connected to a charging circuit 823 and a discharge circuit 825.In various implementations the charging circuit 823includes a power source 826, one or more charging switches 828 and one or more capacitors 830. The power source 826 includes, for instance a battery. The processor 812 initiates a defibrillation shock by causing the power source 826 to charge at least one capacitor among the capacitor(s) 830.For example, the processor(s) 812 activates at least one of the charging switch(es) 828 in the charging circuit 823 to complete a first circuit connecting the power source 826 and the capacitor to be charged. Then, the processor(s) 812 causes the discharge circuit 825 to discharge energy stored in the charged capacitor across a pair of defibrillation electrodes 834, which are in contact with the individual 808. For example, the processor(s) 812 deactivates the charging switch(es) 828 completing the first circuit between the capacitor(s) 830 and the power source 826, and activates one or more discharge switches 832 completing a second circuit connecting the charged capacitor 830 and at least a portion of the individual 808 disposed between defibrillation electrodes 834.

The energy is discharged from the defibrillation electrodes 834 in the form of a defibrillation shock. For example, the defibrillation electrodes 834 are connected to the skin of the individual 808 and located at positions on different sides of the heart of the individual 808, such that the defibrillation shock is applied across the heart of the individual 808. The defibrillation shock, in various examples, depolarizes a significant number of heart cells in a short amount of time. The defibrillation shock, for example, interrupts the propagation of the shockable rhythm (e.g., VF or V-Tach) through the heart. In some examples, the defibrillation shock is 200 J or greater with a duration of about 0.015 seconds. In some cases, the defibrillation shock has a multiphasic (e.g., biphasic) waveform. The discharge switch(es) 832 are controlled by the processor(s) 812, for example. In various implementations, the defibrillation electrodes 834 are connected to defibrillation wires 836. The defibrillation wires 836 are connected to a defibrillation port 838, in implementations. According to various examples, the defibrillation wires 836 are removable from the defibrillation port 838. For example, the defibrillation wires 836 are plugged into the defibrillation port 838.

In various implementations, the processor(s) 812 is operably connected to one or more transceivers 840 that transmit and/or receive data over one or more communication networks 842. For example, the transceiver(s) 840 includes a network interface card (NIC), a network adapter, a local area network (LAN) adapter, or a physical, virtual, or logical address to connect to the various external devices and/or systems. In various examples, the transceiver(s) 840 includes any sort of wireless transceivers capable of engaging in wireless communication (e.g., radio frequency (RF) communication). For example, the communication network(s) 842 includes one or more wireless networks that include a 3^{rd} Generation Partnership Project (3GPP) network, such as a Long Term Evolution (LTE) radio access network (RAN) (e.g., over one or more LTE bands), a New Radio (NR) RAN (e.g., over one or more NR bands), or a combination thereof. In some cases, the transceiver(s) 840 includes other wireless modems, such as a modem for engaging in WI-FI®, WIGIG®, WIMAX®, BLUETOOTH®, or infrared communication over the communication network(s) 842.

The defibrillator 800 is configured to transmit and/or receive data (e.g., ECG data, impedance data, data indicative of one or more detected heart rhythms of the individual 808, data indicative of one or more defibrillation shocks administered to the individual 808, etc.) with one or more external devices 844 via the communication network(s) 842. The external devices 844 include, for instance, the chest-mounted apparatus 819, mobile devices (e.g., mobile phones, smart watches, etc.), Internet of Things (IoT) devices, medical devices, computers (e.g., laptop devices, servers, etc.), or any other type of computing device configured to communicate over the communication network(s) 842. In some examples, the external device(s) 844 is located remotely from the defibrillator 800, such as at a remote clinical environment (e.g., a hospital). According to various implementations, the processor(s) 812 causes the transceiver(s) 840 to transmit data to the external device(s) 844. In some cases, the transceiver(s) 840 receives data from the external device(s) 844 and the transceiver(s) 840 provide the received data to the processor(s) 812 for further analysis.

In various implementations, the external defibrillator 800 also includes a housing 846 that at least partially encloses other elements of the external defibrillator 800. For example, the housing 846 encloses the detection circuit 810, the processor(s) 812, the memory 814, the charging circuit 823, the transceiver(s) 840, or any combination thereof. In some cases, the input device(s) 818 and output device(s) 820 extend from an interior space at least partially surrounded by the housing 846 through a wall of the housing 846. In various examples, the housing 846 acts as a barrier to moisture, electrical interference, and/or dust, thereby protecting various components in the external defibrillator 800 from damage.

In some implementations, the external defibrillator 800 is an automated external defibrillator (AED) operated by an untrained user (e.g., a bystander, layperson, etc.) and can be operated in an automatic mode. In automatic mode, the processor(s) 812 automatically identifies a rhythm in the ECG signal, makes a decision whether to administer a defibrillation shock, charges the capacitor(s) 830, discharges the capacitor(s) 830, or any combination thereof. In some cases, the processor(s) 812 controls the output device(s) 820 to output (e.g., display) a simplified user interface to the untrained user. For example, the processor(s) 812 refrains from causing the output device(s) 820 to display a waveform of the ECG signal and/or the impedance signal to the untrained user, in order to simplify operation of the external defibrillator 800.

In some examples, the external defibrillator 800 is a monitor-defibrillator utilized by a trained user (e.g., a clinician, an emergency responder, etc.) and can be operated in a manual mode or the automatic mode. When the external defibrillator 800 operates in manual mode, the processor(s) 812 cause the output device(s) 820 to display a variety of information that may be relevant to the trained user, such as waveforms indicating the ECG data and/or impedance data, notifications about detected heart rhythms, and the like.

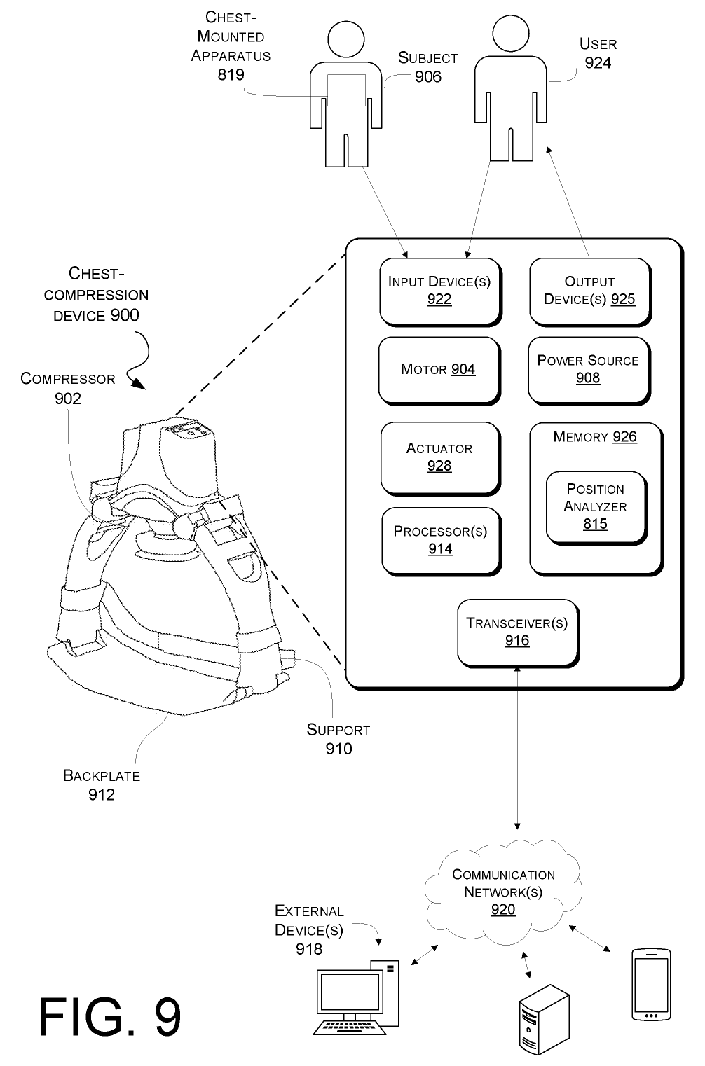

FIG. 9 illustrates a chest compression device 900 configured to perform various functions described herein. For example, the chest compression device 900 is the chest compression device 104 described above with reference to FIG. 1.

In various implementations, the chest compression device 900 includes a compressor 902 that is operatively coupled to a motor 904. The compressor 902 physically administers a force to the chest of a subject 906 that compresses the chest of the subject 906. In some examples, the compressor 902 includes at least one piston that periodically moves between two positions (e.g., a compressed position and a release position) at a compression frequency. For example, when the piston is positioned on the chest of the subject 906, the piston compresses the chest when the piston is moved into the compressed position. A suction cup may be positioned on a tip of the piston, such that the suction cup contacts the chest of the subject 906 during operation. In various cases, the compressor 902 includes a band that periodically tightens to a first tension and loosens to a second tension at a compression frequency. For instance, when the band is disposed around the chest of the subject 906, the band compresses the chest when the band tightens.

The motor 904 is configured to convert electrical energy stored in a power source 908 into mechanical energy that moves and/or tightens the compressor 902, thereby causing the compressor 902 to administer the force to the chest of the subject 906. In various implementations, the power source 908 is portable. For instance, the power source 908 includes at least one rechargeable (e.g., lithium-ion) battery. In some cases, the power source 908 supplies electrical energy to one or more elements of the chest compression device 900 described herein.

In various cases, the chest compression device 900 includes a support 910 that is physically coupled to the compressor 902, such that the compressor 902 maintains a position relative to the subject 906 during operation. In some implementations, the support 910 is physically coupled to a backplate 912, cot, or other external structure with a fixed position relative to the subject 906. According to some cases, the support 910 is physically coupled to a portion of the subject 906, such as wrists of the subject 906.

The operation of the chest compression device 900 may be controlled by at least one processor 914. In various implementations, the motor 904 is communicatively coupled to the processor(s) 914. Specifically, the processor(s) 914 is configured to output a control signal to the motor 904 that causes the motor 904 to actuate the compressor 902. For instance, the motor 904 causes the compressor 902 to administer the compressions to the subject 906 based on the control signal. In some cases, the control signal indicates one or more treatment parameters of the compressions. Examples of treatment parameters include a frequency, timing, depth, force, position, velocity, and acceleration of the compressor 902 administering the compressions. According to various cases, the control signal causes the motor 904 to cease compressions.

In various implementations, the chest compression device 900 includes at least one transceiver 916 configured to communicate with at least one external device 918 over one or more communication networks 920. Any communication network described herein can be included in the communication network(s) 920 illustrated in FIG. 9. The external device(s) 918, for example, includes at least one of a monitor-defibrillator, an AED, an ECMO device, a ventilation device, a patient monitor, a mobile phone, a server, or a computing device. In some implementations, the transceiver(s) 916 is configured to communicate with the external device(s) 918 by transmitting and/or receiving signals wirelessly. For example, the transceiver(s) 916 includes a NIC, a network adapter, a LAN adapter, or a physical, virtual, or logical address to connect to the various external devices and/or systems. In various examples, the transceiver(s) 916 includes any sort of wireless transceivers capable of engaging in wireless communication (e.g., RF communication). For example, the communication network(s) 920 includes one or more wireless networks that include a 3GPP network, such as an LTE RAN (e.g., over one or more LTE bands), an NR RAN (e.g., over one or more NR bands), or a combination thereof. In some cases, the transceiver(s) 916 includes other wireless modems, such as a modem for engaging in WI-FI®, WIGIG®, WIMAX®, BLUETOOTH®, or infrared communication over the communication network(s) 920. The signals, in various cases, encode data in the form of data packets, datagrams, or the like. In some cases, the signals are transmitted as compressions are being administered by the chest compression device 900 (e.g., for real-time feedback by the external device(s) 918), after compressions are administered by the chest compression device 900 (e.g., for post-event review at the external device 918), or a combination thereof.

In various cases, the processor(s) 914 generates the control signal based on data encoded in the signals received from the external device(s) 918. For instance, the signals include an instruction to initiate the compressions, and the processor(s) 914 instructs the motor 904 to begin actuating the compressor 902 in accordance with the signals.

In some cases, the chest compression device 900 includes at least one input device 922. In various examples, the input device(s) 922 is configured to receive an input signal from a user 924, who may be a rescuer treating the subject 906. Examples of the input device(s) 922 include, for instance, at a keypad, a cursor control, a touch-sensitive display, a voice input device (e.g., a microphone), a haptic feedback device (e.g., a gyroscope), or any combination thereof. In various implementations, the processor(s) 914 generate the control signal based on the input signal. For instance, the processor(s) 914 generate the control signal to adjust a frequency of the compressions based on the chest compression device 900 detecting a selection by the user 924 of a user interface element displayed on a touchscreen or detecting the user 924 pressing a button integrated with an external housing of the chest compression device 900.

According to some examples, the input device(s) 922 include one or more sensors. The sensor(s), for example, is configured to detect a physiological parameter of the subject 906. In some implementations, the sensor(s) is configured to detect a state parameter of the chest compression device 900, such as a position of the compressor 902 with respect to the subject 906 or the backplate 912, a force administered by the compressor 902 on the subject 906, a force administered onto the backplate 912 by the body of the subject 906 during a compression, or the like. According to some implementations, the signals transmitted by the transceiver(s) 916 indicate the physiological parameter(s) and/or the state parameter(s).

The chest compression device 900 further includes at least one output device 925, in various implementations. Examples of the output device(s) 925 include, for instance, least one of a display (e.g., a projector, an LED screen, etc.), a speaker, a haptic output device, a printer, or any combination thereof. In some implementations, the output device(s) 925 include a screen configured to display various parameters detected by and/or reported to the chest compression device 900, a charge level of the power source 908, a timer indicating a time since compressions were initiated or paused, and other relevant information.

The chest compression device 900 further includes memory 926. In various implementations, the memory 926 is volatile (such as random access memory (RAM)), non-volatile (such as read only memory (ROM), flash memory, etc.) or some combination of the two. The memory 926 stores instructions that, when executed by the processor(s) 914, causes the processor(s) 914 to perform various operations. In various examples, the memory 926 stores methods, threads, processes, applications, objects, modules, any other sort of executable instruction, or a combination thereof. In some cases, the memory 926 stores files, databases, or a combination thereof. In some examples, the memory 926 includes, but is not limited to, RAM, ROM, EEPROM, flash memory, or any other memory technology. In some examples, the memory 926 includes one or more of CD-ROMs, DVDs, CAM, or other optical storage, magnetic cassettes, magnetic tape, magnetic disk storage or other magnetic storage devices, or any other medium which can be used to store the desired information. In various cases, the memory 926 stores instructions, programs, threads, objects, data, or any combination thereof, that cause the processor(s) 914 to perform various functions. In various cases, the memory 926 stores one or more parameters that are detected by the chest compression device 900 and/or reported to the chest compression device 900.

In implementations of the present disclosure, the memory 926 also stores instructions for executing the position analyzer 815. In various cases, the processor(s) 914, when executing the position analyzer 815, further cause lateral adjustment of the position of the compressor 902 by controlling an actuator 928. For example, the actuator 928 is configured to change the position of the compressor 902 with respect to a superior-inferior direction and/or with respect to a medial-lateral direction.

FIG. 10 illustrates a chest mounted apparatus 1000 may correspond to the chest-mounted apparatus 112, the chest-mounted apparatus 200, the chest-mounted apparatus 400, the chest-mounted apparatus 500, or any combination thereof. In some aspects, the chest mounted apparatus 1000 may be used in combination with a chest compression device such as chest compression device 104. Such a combination may be of two separate devices, or a single device that includes both the chest mounted apparatus 1000 and a chest compression device.

In some aspects, the chest-mounted apparatus 1000 may include a number of electrodes 1010 including defibrillation electrodes that when the apparatus is placed, will be disposed on multiple sides of the heart 1008 of the subject 102. Multiple vectors extend between various pairs of the defibrillation electrodes 1010. As used herein, the term “vector,” and its equivalents, refers to a direction and position of an electrical path extending between multiple electrodes. The vectors, for example, include one or more vectors that extend through at least a portion of the heart 1008 of the subject 102. In various cases, different vectors extending between the defibrillation electrodes 1010 are associated with different efficacies. That is, an electrical shock administered along one vector may successfully treat the condition of the subject 102, whereas an electrical shock administered along a different vector may unsuccessfully treat the condition of the subject 102. Accordingly, it may be beneficial to select a subset of the vectors between the defibrillation electrodes 1010 for therapy administration.

In various implementations, the chest-mounted apparatus 1000 is configured to select, among the vectors, an optimal vector 1012 for administering an electrical therapy to the heart 1008 of the subject 102. In various cases, energy from an electrical signal output along the optimal vector 1012 is predicted to be delivered to the heart 1008 of the subject 102. For instance, the optimal vector 1012 may extend through at least a portion of the heart 1008. One or more techniques can be utilized to identify the optimal vector 1012 among the multiple vectors extending between the defibrillation electrodes 1010.

In some examples, the chest-mounted apparatus 1000 selects the optimal vector 1012 by analyzing feedback from one or more test shocks administered to the defibrillation electrodes 1010. As used herein, the term “test shock,” and its equivalents, refers to an electrical pulse with a defibrillation energy level or a sub-defibrillation energy level. A test shock, in some cases, has a similar shape and/or duration to that of a defibrillation shock. For example, a test shock can be multiphasic (e.g., biphasic). In various examples, a test shock has an energy level in a range of 0.1 J to 200 J, such as a range of 0.1 J to 5 J. In some examples, a test shock is a defibrillating electrical shock. For example, a test shock can have an energy level in a range of 200 J to 360 J.

In some cases, the chest-mounted apparatus 1000 monitors one or more physiological parameters of the subject 102 in response to administration of a test shock along the optimal vector 1012. For example, the chest-mounted apparatus 1000 identifies the optimal vector 1012 by detecting a perturbance in the ECG of the subject 102 in response to the application of the test shock. As used herein, the term “perturbance,” and its equivalents, can refer to a temporary or permanent change. In some cases, the perturbance is a different heart rhythm than the shockable heart rhythm that was present before the test shock. For example, if the ECG indicates that the heart 1008 is in VF prior to the test shock but indicates a non-VF heart rhythm for at least a predetermined time period (e.g., 10 ms to 5 seconds, or 1 to 5 seconds after application of the test shock), then the chest-mounted apparatus 1000 may detect the temporary transition as a perturbance of the VF. The perturbance, for instance, includes one or more of a change in VF rate (e.g., a transient decrease in VF rate), a change in VF amplitude (e.g., a transient increase in peaks within the ECG indicative of VF), a change in AMSA of the ECG, or any combination thereof. In various cases, a perturbance includes one or more temporary changes in the ECG. In various cases, if a test shock causes a perturbance when applied to a pair of the defibrillation electrodes 1010, then the chest-mounted apparatus 1000 infers that the vector extending between the pair of the defibrillation electrodes 1010 is the optimal vector 1012. In contrast, the chest-mounted apparatus 1000 may refrain from detecting a perturbance if the VF continues through the application of the test shock. For instance, if a test shock does not cause a perturbance when applied to a pair of the defibrillation electrodes 1010, then the chest-mounted apparatus 1000 infers that the vector extending between the pair of the defibrillation electrodes 1010 is a non-optimal vector 1014. The test shocks, for example, enable the chest-mounted apparatus 1000 to infer one or more electrical paths that are optimal for terminating a shockable rhythm of the heart 1008. The existence or absence of the perturbance, for instance, is a type of feedback.

In some examples, the chest-mounted apparatus 1000 identifies the optimal vector 1012 and the non-optimal vector 1014 by observing a response to the administration of an electrotherapy across the optimal vector 1012 and the non-optimal vector 1014. For example, the chest-mounted apparatus 1000 may administer a first electrotherapy (e.g., an electrical shock) across the non-optimal vector 1014, wherein the first electrotherapy is configured to treat a medical condition (e.g., VF). The chest-mounted apparatus 1000 , in various cases, analyzes one or more physiological parameters (e.g., ECG) in order to determine whether the medical condition was at least temporarily resolved after the administration of the first electrotherapy. The chest-mounted apparatus 1000 may identify the non-optimal vector 1014 by determining that the medical condition is continuous after the administration of the first electrotherapy. In response to identifying the non-optimal vector 1014, the chest-mounted apparatus 1000 may switch vectors. For instance, the chest-mounted apparatus 1000 outputs a second electrotherapy (e.g., another electrical shock) across the optimal vector 1012. The chest-mounted apparatus 1000 may, for instance, identify the optimal vector 1012 by determining that the condition is at least temporarily resolved by the administration of the second electrotherapy.

Some implementations of the present disclosure enable the selection of the optimal vector 1012 for the purposes of administering a pacing therapy. In various cases, the chest-mounted apparatus 1000 identifies the optimal vector 1012 by determining that pacing pulses output along the optimal vector 1012 result in electrical capture of the heart 1008. In particular examples, the chest-mounted apparatus 1000 detects that the subject 102 has bradycardia. In response, the chest-mounted apparatus 1000 outputs first pacing pulses to electrodes associated with a first vector (e.g., the non-optimal vector). In response to administering the first pacing pulses, the chest-mounted apparatus 1000 analyzes one or more physiological parameters of the subject 102 in order to determine whether the first pacing pulses successfully resulted in electrical capture of the heart 1008. In some cases, the chest-mounted apparatus 1000 inputs one or more of an ECG feature (e.g., a T-wave or QRS complex), a blood oxygenation, a plethysmographic waveform (e.g., sampled from a pulse oximeter), a magnitude of oscillation in a detected plethysmographic waveform, a blood pressure, or an EtCO₂ of the subject 102 into a computing model configured to calculate a likelihood that capture has been achieved. Techniques for automatically identifying pacing capture are described, for instance, in US Pub. No. 2022/0219000, which is incorporated by reference herein in its entirety. In some cases, chest-mounted apparatus 1000 determines that the first vector is the non-optimal vector 1014 by determining that the first pacing pulses do not result in electrical capture of the heart 1008. In response to determining that the first vector is the non-optimal vector 1014, the chest-mounted apparatus 1000 may administer second pacing pulses to a second vector that is different than the first vector. For example, the chest-mounted apparatus 1000 may determine that the second vector is the optimal vector 1012 in response to determining that the second pacing pulses result in electrical capture of the heart 1008.

In various cases, once the chest-mounted apparatus 1000 identifies the optimal vector 1012 based on a first type of electrotherapy, the chest-mounted apparatus 1000 is configured to apply a second type of electrotherapy across the same optimal vector 1012. For example, if chest-mounted apparatus 1000 identifies the optimal vector 1012 by detecting electrical capture in response to pacing pulses being applied to the subject 102 across the optimal vector 1012, chest-mounted apparatus 1000 may select the optimal vector 1012 for the application of an electrical shock if the subject 102 subsequently exhibits VF.

In some cases, the chest-mounted apparatus 1000 is further configured to identify the optimal vector 1012 and/or the non-optimal vector 1014 by comparing transthoracic impedances associated with the vectors between the defibrillation electrodes 1010. A transthoracic impedance, for instance, is detected by applying an electrical signal across a pair of the defibrillation electrodes 1010, detecting an electrical signal resulting from the applied electrical signal, and determining the impedance based on the applied and detected electrical signals. For example, various parts of the heart 1008 (e.g., atria and ventricles) expand and/or contract during the time period in which the transthoracic impedance is detected. If one or more portions of the heart 1008 are located along the electrical path between the pair of defibrillation electrodes 1010, then the transthoracic impedance signal will change over time in accordance with the movement of the heart 1008. Thus, the optimal vector 1012 and/or the non-optimal vector 1014, in some cases, are identified based on variances in the transthoracic impedances along the different vectors.

In some cases, an analysis of transthoracic impedances can enable the chest-mounted apparatus 1000 to select between multiple adequate vectors. For example, if the chest-mounted apparatus 1000 determines that the peak amplitudes of two ECG leads, associated with a first vector and a second vector, are above at least one threshold, the chest-mounted apparatus 1000 may select the first vector as the optimal vector 1012 by determining that a transthoracic impedance associated with the first vector is lower than a transthoracic impedance associate with the second vector. For instance, the lower transthoracic impedance associated with the first vector may indicate that the electrical path between the pair of defibrillation electrodes 1010 associated with the first vector is more targeted toward the heart 1008 than the electrical path between the pair of defibrillation electrodes 1010 associated with the second vector.

The chest-mounted apparatus 1000 is configured to recommend and/or cause a treatment to be applied across the optimal vector 1012. In some examples, the chest-mounted apparatus 1000 refrains from applying the treatment across the non-optimal vector 1014. For instance, upon identifying the optimal vector 1014, the chest-mounted apparatus may recommend, or apply, at least one electrical shock to the defibrillation electrodes 1010 associated with the optimal vector 1012. In various cases, the selection of the optimal vector 1012 enhances the likelihood that the treatment will be successful. For instance, an electrical shock applied to the optimal vector 1012 may have a greater likelihood of resolving VF of the heart 1008 than an electrical shock applied to the non-optimal vector 1014.

Although FIG. 10 illustrates a single optimal vector 1012 and a single non-optimal vector 1014, implementations are not so limited. For example, in some cases, multiple optimal vectors and/or multiple non-optimal vectors are identified by the chest-mounted apparatus 1000. In some implementations, the chest-mounted apparatus 1000 outputs respective therapies to respective combinations of the defibrillation electrodes 1010 corresponding to the multiple optimal vectors. For instance, the chest-mounted apparatus 1000 outputs a first electrical shock to a first optimal vector, and subsequently outputs a second electrical shock to a second optimal vector if the subject 102 refibrillates after administration of the first electrical shock. That is, in some cases, the chest-mounted apparatus 1000 rotates therapies among multiple optimal vectors associated with various combinations of the defibrillation electrodes 1010. According to some cases, the change in vectors may enhance the likelihood that the condition of the heart 1008 of the subject 102 will be resolved.

In some cases, the chest-mounted apparatus 1000 administers, at least in part, a multi-shock (e.g., double-sequential defibrillation (DSD)) therapy along the optimal vector 1012. The chest-mounted apparatus 1000 may determine that the subject 102 has a condition that warrants the multi-shock therapy. For instance, the chest-mounted apparatus 1000 may detect that the subject 102 has VF that has not responded to one or more previous defibrillation shocks administered to the subject 102 (e.g., refractory VF). In response to detecting the condition that warrants the multi-shock therapy, the chest-mounted apparatus 1000 may administer to the subject 102, multiple sequential electrical shocks along the optimal vector 1012 or multiple optimal vectors. For example, the chest-mounted apparatus 1000 may administer a first electrical shock along the optimal vector 1012, a second electrical shock along another optimal vector, wherein the first electrical shock and the second electrical shock temporally overlap. In some cases, the chest-mounted apparatus 1000 includes multiple therapy circuits, each with a respective capacitor, such that the chest-mounted apparatus 1000 may output the multiple electrical shocks along the different vectors. For instance, the chest-mounted apparatus 1000 outputs multiple electrical shocks along different paths through different combinations of the defibrillation electrodes 1010.

Although not illustrated in FIG. 10, any of the vectors described herein can include virtual vectors. As used herein, the term “virtual vector,” and its equivalents, can refer to a vector that results from a combination of more than two electrodes. For example, a virtual vector may extend from a first electrode among the defibrillation electrodes 1010 to second and third electrodes among the defibrillation electrode 1010, such that the virtual vector extends along a position between the second and third electrodes. A test shock, an electrical therapy, or other type of electrical signal can be applied to the virtual vector by activating the first, second, and third electrodes, for instance.

### EXAMPLE CLAUSES

The following clauses provide various examples of implementations of the present disclosure:

A system, including: a chest-mounted apparatus including: a flexible housing configured to be disposed on a chest of a subject; an adhesive configured to adhere the flexible housing to the chest of the subject; and a circuit including an array of capacitive sensors integrated with the flexible housing, the array of capacitive sensors being configured to detect a position of a compression applied to the chest of the subject by a compressor and along a plane that is normal to an anterior-posterior direction; and a medical device communicatively coupled with the chest-mounted apparatus and including: a display; and a processor configured to: determine that the position of the compression is outside of a predetermined target range; and in response to determining that the position of the compression is outside of the predetermined target range, cause the display to visually present an instruction to reposition the compressor.

The system of clause 1, wherein the flexible housing further includes a visual marker indicating the predetermined target range, and wherein the array of capacitive sensors includes a first sensor within the predetermined target range and a second sensor outside of the predetermined target range.

The system of clause 1 or 2, wherein the chest-mounted apparatus further includes: a first electrode integrated with the flexible housing and configured to be applied to an anterior position on the chest of the subject; a second electrode integrated with the flexible housing and configured to be applied to a lateral position on the chest of the subject; a connector configured to be removably connected to a port of the medical device; and a cable electrically connected with the first electrode, the second electrode, and the connector, wherein the medical device is a defibrillator further including: a detection circuit configured to detect an electrical signal indicative of an electrocardiogram (ECG) of the subject; and a treatment circuit including a capacitor, and wherein the processor is further configured to: determine that the ECG is indicative of ventricular fibrillation (VF); and in response to determining that the ECG is indicative of VF, cause the treatment circuit to discharge the capacitor to the first electrode and the second electrode via the port, the connector, and the cable.

An apparatus, including: a housing configured to be disposed on a chest of a subject; and a circuit including an array of sensors integrated with the housing, the array of sensors being configured to detect a position of a compression applied to the chest of the subject along a plane that is normal to an anterior-posterior direction.

The apparatus of clause 4, wherein the housing includes a flexible material.

The apparatus of clause 4 or 5, wherein the housing includes a material with a coefficient of friction that is about 0.5 or greater.

The apparatus of any of clauses 4 to 6, wherein the array of sensors include capacitive sensors, resistive sensors, piezoelectric sensors, or triboelectric sensors.

The apparatus of any of clauses 4 to 7, wherein the array of sensors includes a first sensor and a second sensor separated from each other along a superior-inferior direction.

The apparatus of any of clauses 4 to 8, wherein the array of sensors includes a first sensor and a second sensor separated from each other along a medial-lateral direction.

The apparatus of any of clauses 4 to 9, further including: an adhesive disposed on the housing, the adhesive being configured to adhere the apparatus to the chest of the subject.

The apparatus of any of clauses4 to 10, further including: a connector configured to be removably connected to a medical device; and a cable electrically connected with the circuit and the connector, the cable being configured to transmit, from the circuit to the connector, a communication signal indicative of the position of the compression applied to the chest of the subject.

The apparatus of any of clauses 4 to 11, further including: a first electrode integrated with the housing and configured to be applied to an anterior position on the chest of the subject; a second electrode integrated with the housing and configured to be applied to a lateral position on the chest of the subject; a connector configured to be removably connected to a medical device; and a cable electrically connected with the first electrode, the second electrode, and the connector, the cable being configured to transmit, from the connector to the first electrode and the second electrode, an electrical signal, wherein the first electrode and the second electrode are configured to output the electrical signal as an electrical shock.

A method, including: detecting, by a sensor among an array of sensors in a chest-mounted apparatus, a compression applied to a chest of a subject by a compressor; determining, by analyzing a position of the sensor among the array of sensors, a position of the compression along a plane that is normal to an anterior-posterior direction; determining that the position of the compression is outside of a predetermined target range; and in response to determining that the position of the compression is outside of the predetermined target range, outputting an instruction to reposition the compressor.

The method of clause 13, wherein detecting, by the sensor among the array of sensors in the chest-mounted apparatus, the compression applied to the chest of the subject by the compressor includes: detecting a change in a capacitance or a resistance of an element of the sensor.

The method of clause 13 or 14, wherein detecting, by the sensor among the array of sensors in the chest-mounted apparatus, the compression applied to the chest of the subject by the compressor includes: detecting an electrical signal generated by the sensor.

The method of any of clauses 13 to 15, wherein outputting the instruction to reposition the compressor includes visually presenting the instruction or audibly presenting the instruction.

The method of any of clauses 13 to 16, further including: detecting a physiological parameter of the subject, the physiological parameter being indicative of blood circulation in the subject, wherein determining that the position of the compression is outside of the predetermined target range further includes determining that the physiological parameter is outside of a threshold range.

The method of clause 17, wherein the physiological parameter includes a blood pressure, a partial pressure of CO₂ in an airway of the subject, a blood oxygenation of the subject, or a blood flow rate of the subject.

The method of any of clauses 13 to 18, further including: determining that an electrocardiogram (ECG) of the subject is indicative of a shockable arrhythmia; and in response to determining that the ECG of the subject is indicative of the shockable arrhythmia, outputting an electrical shock to electrodes integrated with the chest-mounted apparatus.

The method of clause 19, further including: determining a size of the subject; and selecting, among multiple pairs of electrodes integrated with the chest-mounted apparatus, the electrodes by analyzing the size of the subject.

A system, including: a chest-mounted apparatus including: a flexible housing configured to be disposed on a chest of a subject; an adhesive disposed on a lower surface of the flexible housing, the adhesive being configured to adhere the flexible housing to the chest of the subject; and a circuit including an array of capacitive sensors integrated with the flexible housing, the array of capacitive sensors being configured to detect a position of a compression applied to the flexible housing; and a mechanical chest compression device communicatively coupled with the circuit, the mechanical chest compression device including: a compressor configured to apply the compression to an upper surface of the flexible housing; a motor configured to cause the compressor to apply the compression to the upper surface of the flexible housing; an actuator configured to reposition the compressor along a direction that is parallel to the upper surface of the flexible housing; and a processor configured to: determine that the position of the compression is outside of a target area; and in response to determining that the position of the compression is outside of the target area, cause the actuator to reposition the compressor within the target area.

The system of clause 21, wherein the upper surface of the flexible housing of the chest-mounted apparatus is configured to be removably coupled with the compressor of the mechanical chest compression device.

The system of clause 21 or 22, further including: a monitor communicatively coupled with the mechanical chest compression device and configured to detect a blood pressure of the subject, a partial pressure of CO₂ in an airway of the subject, a blood oxygenation of the subject, or a blood flow rate of the subject, wherein the processor is configured to determine that the position of the compression is outside of the target area by determining that the blood pressure, the partial pressure of CO₂, the blood oxygenation, or the blood flow rate is below a threshold.

A medical device, including: an input device configured to receive an indication of a position of a first compression applied to a surface of a chest-mounted apparatus; and a processor configured to: determine that the position of the first compression is outside of a target area; and in response to determining that the position of the first compression is outside of the target area, output an instruction to apply a second compression to the surface of the chest-mounted apparatus at a different position than the position of the first compression or to prevent administration of the second compression.

The medical device of clause 24, wherein the input device includes a port or a transceiver configured to receive a communication signal indicating the position of the first compression.

The medical device of clause 24 or 25, wherein the input device includes an array of sensors integrated with a housing of the chest-mounted apparatus, the array of sensors including capacitive sensors, resistive sensors, piezoelectric sensors, or triboelectric sensors.

The medical device of any of clauses 24 to 26, further including: a compressor configured to administer the first compression and the second compression to the chest-mounted apparatus; and an actuator configured to reposition the compressor along the surface of the chest-mounted apparatus in response to the instruction.

The medical device of clause 27, wherein the input device includes a sensor integrated with the compressor, the sensor being configured to detect a distance between the sensor and a fiducial marker disposed in the chest-mounted apparatus, the fiducial marker being disposed within the target area.

The medical device of clause 27 or 28, wherein the compressor is configured to be removably coupled with the surface of the chest-mounted apparatus.

The medical device of any of clauses 24 to 29, further including: a physiological parameter sensor configured to detect a physiological parameter of a subject that is indicative of blood circulation, the physiological parameter including a blood pressure, a pulse rate, a blood flow rate, a blood oxygenation, or a partial pressure of CO₂ in an airway of the subject, wherein the processor is configured to determine that the position of the first compression is outside of the target area by determining that the physiological parameter is below a threshold.

The medical device of any of clauses 24 to 30, the position being a first position, further including: a physiological parameter sensor configured to detect a physiological parameter of a subject that is indicative of blood circulation, the physiological parameter including a blood pressure, a pulse rate, a blood flow rate, a blood oxygenation, or a partial pressure of CO₂ in an airway of the subject, wherein the processor is configured to determine that the position of the first compression is outside of the target area by: determining a change in the physiological parameter between a first time period and a second time period, a third compression being administered at a second position during the first time period, the first compression being administered during the second time period, the second time period occurring after the first time period; and determining that the change in the physiological parameter is outside of a threshold range.

The medical device of any of clauses 24 to 31, wherein the different position is closer to a center of the surface of the chest-mounted apparatus than the position of the first compression.

The medical device of any of clauses 24 to 32, further including: an output device configured to output the instruction to a user.

A method, including: identifying a position of a first compression on a surface of a chest-mounted apparatus; determining that the position of the first compression on the surface of the chest-mounted apparatus is outside of a target area; and in response to determining that the position of the first compression on the surface of the chest-mounted apparatus is outside of the target area, applying a second compression to a different position on the surface of the chest-mounted apparatus than the position of the first compression.

The method of clause 34, wherein identifying the position of the first compression on the surface of the chest-mounted apparatus includes: detecting, by an array of sensors integrated with the chest-mounted apparatus, the position of the first compression.

The method of clause 34 or 35, wherein the first compression is applied to the chest-mounted apparatus by a compressor, and wherein identifying the position of the first compression on the surface of the chest-mounted apparatus includes: detecting, by a sensor integrated with the compressor, a distance between the compressor and a fiducial marker integrated with the chest-mounted apparatus.

The method of clause 36, wherein applying the second compression to the different position on the surface of the chest-mounted apparatus than the position of the first compression includes: repositioning, by an actuator, the compressor; and applying the second compression using the compressor.

The method of any of clauses34 to 37, wherein determining that the position of the first compression on the surface of the chest-mounted apparatus is outside of the target area includes determining that a physiological parameter of a subject is outside of a threshold range.

The method of clause 38, further including: in response to applying the second compression to the different position on the surface of the chest-mounted apparatus, determining that the physiological parameter has entered the threshold range.

The method of any of clauses 34 to 39, further including: outputting, to a user, an instruction to reposition a medical device performing the method with respect to the surface of the chest-mounted apparatus, wherein applying the second compression is in response to outputting the instruction to reposition the medical device.

The method of clause 40, wherein the medical device includes a mechanical chest compression device.

### CONCLUSION

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be used for realizing implementations of the disclosure in diverse forms thereof.

As will be understood by one of ordinary skill in the art, each implementation disclosed herein can comprise, consist essentially of or consist of its particular stated element, step, or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the implementation to the specified elements, steps, ingredients or components and to those that do not materially affect the implementation. As used herein, the term “based on” is equivalent to “based at least partly on,” unless otherwise specified.

Unless otherwise indicated, all numbers expressing quantities, properties, conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing implementations (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate implementations of the disclosure and does not pose a limitation on the scope of the disclosure. No language in the specification should be construed as indicating any non-claimed element essential to the practice of implementations of the disclosure.

Groupings of alternative elements or implementations disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain implementations are described herein, including the best mode known to the inventors for carrying out implementations of the disclosure. Of course, variations on these described implementations will become apparent to those of ordinary skill in the art upon reading the foregoing description. The inventors expect skilled artisans to employ such variations as appropriate, and the inventors intend for implementations to be practiced otherwise than specifically described herein. Accordingly, the scope of this disclosure includes all modifications and equivalents of the subject matter recited in the claims appended hereto as permitted by applicable law. Moreover, any combination of the above-described elements in all possible variations thereof is encompassed by implementations of the disclosure unless otherwise indicated herein or otherwise clearly contradicted by context.

What is claimed is:

1. A system, comprising:
a chest-mounted apparatus comprising:
a flexible housing configured to be disposed on a chest of a subject;
an adhesive configured to adhere the flexible housing to the chest of the subject; and
a circuit comprising an array of capacitive sensors integrated with the flexible housing, the array of capacitive sensors being configured to detect a position of a compression applied to the chest of the subject by a compressor and along a plane that is normal to an anterior-posterior direction; and
a medical device communicatively coupled with the chest-mounted apparatus and comprising:
a display; and
a processor configured to:
determine that the position of the compression is outside of a predetermined target range; and
in response to determining that the position of the compression is outside of the predetermined target range, cause the display to visually present an instruction to reposition the compressor.

2. The system of claim 1, wherein the flexible housing further comprises a visual marker indicating the predetermined target range, and
wherein the array of capacitive sensors comprises a first sensor within the predetermined target range and a second sensor outside of the predetermined target range.

3. The system of claim 1, wherein the chest-mounted apparatus further comprises:
a first electrode integrated with the flexible housing and configured to be applied to an anterior position on the chest of the subject;
a second electrode integrated with the flexible housing and configured to be applied to a lateral position on the chest of the subject;
a connector configured to be removably connected to a port of the medical device; and
a cable electrically connected with the first electrode, the second electrode, and the connector,
wherein the medical device is a defibrillator further comprising:
a detection circuit configured to detect an electrical signal indicative of an electrocardiogram (ECG) of the subject; and
a treatment circuit comprising a capacitor, and
wherein the processor is further configured to:
determine that the ECG is indicative of ventricular fibrillation (VF); and
in response to determining that the ECG is indicative of VF, cause the treatment circuit to discharge the capacitor to the first electrode and the second electrode via the port, the connector, and the cable.

4. An apparatus, comprising:
a housing configured to be disposed on a chest of a subject; and
a circuit comprising an array of sensors integrated with the housing, the array of sensors being configured to detect a position of a compression applied to the chest of the subject along a plane that is normal to an anterior-posterior direction.

5. The apparatus of claim 4, wherein the housing comprises a flexible material.

6. The apparatus of claim 4, wherein the housing comprises a material with a coefficient of friction that is about 0.5 or greater.

7. The apparatus of claim 4, wherein the array of sensors comprise capacitive sensors, resistive sensors, piezoelectric sensors, or triboelectric sensors.

8. The apparatus of claim 4, wherein the array of sensors comprises a first sensor and a second sensor separated from each other along a superior-inferior direction.

9. The apparatus of claim 4, wherein the array of sensors comprises a first sensor and a second sensor separated from each other along a medial-lateral direction.

10. The apparatus of claim 4, further comprising:
an adhesive disposed on the housing, the adhesive being configured to adhere the apparatus to the chest of the subject.

11. The apparatus of claim 4, further comprising:
a connector configured to be removably connected to a medical device; and
a cable electrically connected with the circuit and the connector, the cable being configured to transmit, from the circuit to the connector, a communication signal indicative of the position of the compression applied to the chest of the subject.

12. The apparatus of claim 4, further comprising:
a first electrode integrated with the housing and configured to be applied to an anterior position on the chest of the subject;
a second electrode integrated with the housing and configured to be applied to a lateral position on the chest of the subject;
a connector configured to be removably connected to a medical device; and
a cable electrically connected with the first electrode, the second electrode, and the connector, the cable being configured to transmit, from the connector to the first electrode and the second electrode, an electrical signal,
wherein the first electrode and the second electrode are configured to output the electrical signal as an electrical shock.

13. A method, comprising:
detecting, by a sensor among an array of sensors in a chest-mounted apparatus, a compression applied to a chest of a subject by a compressor;
determining, by analyzing a position of the sensor among the array of sensors, a position of the compression along a plane that is normal to an anterior-posterior direction;
determining that the position of the compression is outside of a predetermined target range; and
in response to determining that the position of the compression is outside of the predetermined target range, outputting an instruction to reposition the compressor.

14. The method of claim 13, wherein detecting, by the sensor among the array of sensors in the chest-mounted apparatus, the compression applied to the chest of the subject by the compressor comprises:
detecting a change in a capacitance or a resistance of an element of the sensor.

15. The method of claim 13, wherein detecting, by the sensor among the array of sensors in the chest-mounted apparatus, the compression applied to the chest of the subject by the compressor comprises:
detecting an electrical signal generated by the sensor.

16. The method of claim 13, wherein outputting the instruction to reposition the compressor comprises visually presenting the instruction or audibly presenting the instruction.

17. The method of claim 13, further comprising:
detecting a physiological parameter of the subject, the physiological parameter being indicative of blood circulation in the subject,
wherein determining that the position of the compression is outside of the predetermined target range further comprises determining that the physiological parameter is outside of a threshold range.

18. The method of claim 17, wherein the physiological parameter comprises a blood pressure, a partial pressure of CO₂ in an airway of the subject, a blood oxygenation of the subject, or a blood flow rate of the subject.

19. The method of claim 13, further comprising:
determining that an electrocardiogram (ECG) of the subject is indicative of a shockable arrhythmia; and
in response to determining that the ECG of the subject is indicative of the shockable arrhythmia, outputting an electrical shock to electrodes integrated with the chest-mounted apparatus.

20. The method of claim 19, further comprising:
determining a size of the subject; and
selecting, among multiple pairs of electrodes integrated with the chest-mounted apparatus, the electrodes by analyzing the size of the subject.
